(19) United States
(12) Patent Application Publication
(10) Pub. No.: US 2019/0350889 A1
(43) Pub. Date: Nov. 21, 2019

(54) Compositions And Methods Of Use Of Phorbol Esters In The Treatment Of Parkinson's Disease

(71) Applicant: Richard L. CHANG, Laguna Woods, CA (US)

(72) Inventor: Richard L. CHANG, Laguna Woods, CA (US)

(21) Appl. No.: 16/347,793

(22) PCT Filed: Nov. 9, 2017
(86) PCT No.: PCT/US17/60729

§ 371 Date: May 6, 2019

Related U.S. Application Data
(60) Provisional application No. 62/420,177, filed on Nov. 10, 2016.

(51) Int. Cl. A61K 31/216; A61P 25/16

(52) U.S. Cl. CPC A61K 31/216; A61P 25/16

(57) ABSTRACT

Methods and compositions containing a phorbol ester or a derivative of a phorbol ester are provided for the treatment of chronic and progressive conditions such as Parkinson's disease. Such conditions may be caused by disease, be symptoms or sequelae of disease. Additional compositions and methods are provided which employ a phorbol ester or derivative compound in combination with at least one additional agent to yield more effective treatment tools against acute and chronic conditions in mammalian subjects.

### ADDITIONAL DISCLOSURES

Additional disclosures relating to the instant application may be found in “Compositions And Methods Of Use Of Phorbol Esters” U.S. patent application Ser. No. 12/023,753, filed Jan. 31, 2008, PCT Patent Application Serial No. PCT/US08/01299, filed Jan. 31, 2008, to Richard L. Chang, et al. which claims priority benefit of U.S. Provisional Patent Application Ser. No. 60/898,810, filed Jan. 31, 2007. U.S. Continuation patent application Ser. No. 13/595,072, filed Aug. 27, 2012, U.S. Continuation patent application Ser. No. 13/794,467, filed Mar. 11, 2013, U.S. Continuation patent application Ser. No. 13/999,347, filed Feb. 13, 2014; “Compositions And Methods Of Use Of Phorbol Esters In The Treatment Of Neoplasms” U.S. patent application Ser. No. 13/745,745, filed Jan. 18, 2013, to Richard L. Chang, et al, which claims priority benefit of U.S. Provisional Patent Application Ser. No. 61/588,165, filed Jan. 18, 2012, U.S. Continuation patent application Ser. No. 14/025,206, filed Sep. 12, 2013, U.S. Continuation patent application Ser. No. 14/026,473, filed Sep. 13, 2013, U.S. Continuation patent application Ser. No. 13/999,339, filed Feb. 13, 2014; “Compositions And Methods Of Use Of Phorbol Esters In The Treatment Of Stroke” U.S. patent application Ser. No. 13/745,742, filed Jan. 18, 2013, PCT Patent Application Serial No. PCT/US2013/022325, filed Jan. 18, 2013, to Richard L. Chang, et al, which claims priority benefit of U.S. Provisional Patent Application Ser. No. 61/588,167, filed Jan. 18, 2012, U.S. Continuation patent application Ser. No. 14/025,176, filed Sep. 12, 2013, U.S. Continuation patent application Ser. No. 14/026,534, filed Sep. 13, 2013, U.S. Continuation patent application Ser. No. 13/999,331, filed Feb. 13, 2014; “Compositions And Methods Of Use Of Phorbol Esters” U.S. Continuation-In-Part patent application Ser. No. 13/745,740, filed Jan. 18, 2013, PCT Patent Application Serial No. PCT/US2013/022324, filed Jan. 18, 2013, to Richard L. Chang, et al., which claims priority benefit of U.S. Provisional Patent Application Ser. No. 61/588,162, filed Jan. 18, 2012, U.S. Continuation patent application Ser. No. 14/025,163, filed Sep. 12, 2013, U.S. Continuation-In-Part patent application Ser. No. 14/027,320, filed Sep. 16, 2013, and U.S. Continuation patent application Ser. No. 13/999,332, filed Feb. 13, 2014, each of which is incorporated herein by reference in its entirety for all purposes.

### TECHNICAL FIELD

The present invention relates to compositions and methods for the treatment of Parkinson's disease and the symptoms of Parkinson's disease. Specifically, the present invention relates to the use of phorbol esters in the treatment and prevention of Parkinson's disease and related symptoms.

### BACKGROUND

Parkinson's disease is a progressive, degenerative neurological disorder affecting 1 in 500 people. It is believed that between 7 and 10 million people worldwide are afflicted with Parkinson's disease. Approximately 60,000 new cases per year are clinically diagnosed in the United States alone. Parkinson's disease and its symptoms are a result of neuronal death in an area of the brain known as the substantia nigra which controls voluntary movement, plays a role in the production of dopamine and also regulates mood. The substantia nigra is located in a part of the mid brain (mesencephalon) called the basal ganglia. The specific etiology of Parkinson's disease is not well understood although much is known about the biological functions that are affected. It is believed that Parkinson's disease occurs as a result of both environmental and genetic factors including but not limited to oxidative stress, mitochondrial dysfunction, amino acid imbalances, protein misfolding, immune system reactions, vitamin and mineral deficiencies, lipid peroxidation, the presence of lewy bodies and other types of undiagnosed medical disease.

The main pathologic feature of Parkinson's disease is the loss of dopaminergic cells in the basal ganglia, especially in the substantia nigra which leads to progressive loss of control over voluntary movement, speech and other autonomic dysfunction. While various therapies have been attempted since the late 1800's, there is no cure for Parkinson's disease, only symptomatic treatment and the condition is considered chronic as well as being progressive. Drugs currently used in the treatment of Parkinson's disease can help control some of the symptoms but may lose their effectiveness over time as well as having deleterious side effects.

Plants have historically served many medicinal purposes. The World Health Organization (WHO) estimates that 4 billion people, 80% of the world population, presently use herbal medicine for some aspect of primary health care. (WHO Fact sheet Fact sheet No 134 December 2008) However, it can be difficult to isolate the specific compound that has the curative effect and reproduce it on a commercial scale. Additionally, while active compounds may be isolated from a plant, the other parts of a plant such as minerals, vitamins, volatile oils, glycosides, alkaloids, bioflavonoids and other inert or unidentified compounds may not be clearly identified or listed in their therapeutic use. Other substances may also be involved in the functioning of the active ingredient or creating the medicinal effect for which the plant is known making the use, purification and commercialization of plant based pharmaceutical agents a challenge.

Phorbol is a naturally derived plant based organic compound of the tigliane family of diterpenes. It was first isolated in 1934 as a hydrolysis product of Croton oil derived from the seeds of Croton tiglium, a leafy shrub of the Euphorbiaceae family that is native to Southeastern Asia. Various esters of phorbol have demonstrated important biological properties including the reported ability to mimic diacylglycerols by activating protein kinase C (PKC) and modulating downstream cell signaling pathways (including the mitogen-activated protein kinase (MAPK) pathways). Phorbol esters are additionally thought to bind to chimaerins, the Ras activator RasGRP, and the vesicle-priming protein Munc-13 (Brose N. Rosenmund C., J Cell Sci; 115:4399-411 (2002)). Some phorbol esters also induce nuclear factor-kappa B (NF-κB). The most notable physiological property of phorbol esters is their reported capacity to act as tumor promoters. (Blumberg, 1988; Goel, G et al., Int. Journal of Toxicology 26, 279-288 (2007)).

12-O-tetradecanoylphorbol-13-acetate (TPA), also called phorbol-12-myristate-13-acetate (PMA), is a phorbol ester used in models of carcinogenesis as an inducer for differentiation and/or apoptosis in multiple cell lines and primary cells. TPA has also been reported to cause an increase in circulating white blood cells and neutrophils in patients whose bone marrow function has been depressed by chemotherapy (Han Z. T. et al. Proc. Natl. Acad. Sci. 95, 5363-5365 (1998)) and inhibit the HIV-cytopathic effects on MT-4 cells. (Mekkawy S. et al., Phytochemistry 53, 47-464 (2000)). However, due to a variety of factors, including caustic reactions when contacted with the skin and concerns for its potential toxicity, TPA has not been shown to be an effective tool in the treatment of diseases. Indeed, as phorbol esters play a key role in activation of protein kinase C (PKC), which triggers various cellular responses resulting in inflammatory responses and tumor development (Goel et al., Int. Journal of Toxicology 26, 279-288 (2007)), phorbol esters would generally be excluded from possible treatment candidates for inflammatory diseases or conditions that involve inflammatory reactions such as stroke or Parkinson's Disease. However, treatment with retinoic acid followed by phorbol ester 12-O-tetradecanoylphorbol-13-acetate results in a DAergic neuronal phenotype and decreases the susceptibility of cells to neurotoxins and neuroprotective agents.

(Xie, H., Hu, L. & Li, G. SH-SY5Y human neuroblastoma cell line: in vitro cell model of dopaminergic neurons in Parkinson's disease. Chin. Med. J. 123, 1086-1092, 2010).

Treatment for Parkinson's disease is currently symptomatic, there is no one treatment that is effective for all symptoms of Parkinson's disease. There is an essential need for new and more effective treatments for individuals suffering from Parkinson's disease and other related disorders.

### SUMMARY

The present invention relates to a compositions and methods for treating Parkinson's disease using phorbol esters. These compositions and methods are effective in treating chronic or potentially recurring conditions, or to repair the damage caused by acute cases of chronic diseases such as Parkinson's Disease. Symptoms of Parkinson's disease that may be treated or prevented by the use of the compositions and methods described herein include, but are not limited to, tremor at rest, stiffness, bradykinesia, rigidity, speech impairment, a feeling of weakness in the limbs, lack of dexterity, poor coordination, imbalance, cognitive impairment, dementia, mood impairment, drowsiness, sleep disturbances, autonomic dysfunction and postural instability.

There is currently is no specific test for Parkinson's disease, although recent MRI findings have indicated the appearance of a ‘swallow Tail’ in a weighted 3T image have concluded that: The healthy nigrosome-1 can be readily depicted on high-resolution 3T-SWI giving rise to a ‘swallow tail’ appearance of the dorsolateral substantia nigra, and this feature is lost in PD. Visual radiological assessment yielded a high diagnostic accuracy for PD vs. an unselected clinical control population. Assessing the substantia nigra on SWI for the typical ‘swallow tail’ appearance has potential to become a new and easy applicable 3T MRI diagnostic tool for nigra degeneration in PD. (Schwarz S T, Afzal M, Morgan P S, Bajaj N, Gowland P A, et al. (2014) The ‘Swallow Tail’ Appearance of the Healthy Nigrosome—A New Accurate Test of Parkinson's Disease: A Case-Control and Retrospective Cross-Sectional MRI Study at 3T. PLoS ONE 9(4): e93814). If this is adopted and accepted, it will offer a clinical biomarker to accurately assess the efficacy of new treatments in Parkinson's disease. Currently successful treatment will be determined according to the accepted conventional method based on the Unified Parkinson's Disease Rating Scale (UPDRS) including decreases in tremor at rest, stiffness, bradykinesia, rigidity, speech impairment, a feeling of weakness in the limbs, lack of dexterity, poor coordination, imbalance, cognitive impairment, dementia, mood impairment, drowsiness, sleep disturbances, autonomic dysfunction and postural instability. The compositions and methods described herein achieve the foregoing and satisfy additional objects and advantages by providing novel and surprisingly effective methods and compositions for modulating cell signaling pathways and/or treating diseases and symptoms of diseases or conditions using compositions containing a phorbol ester or derivative composition of the Formula I, below:

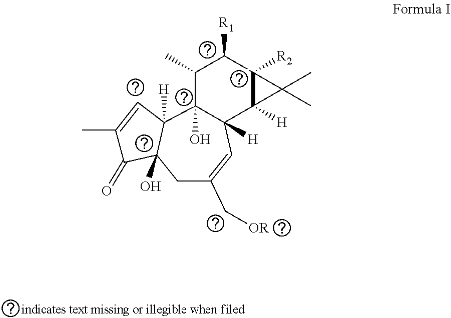

wherein R₁ and R₂ may be hydrogen; hydroxyl;

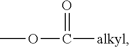

wherein the alkyl group contains 1 to 15 carbon atoms;

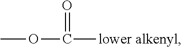

wherein a lower alkenyl group contains between 1 to 7 carbon atoms;

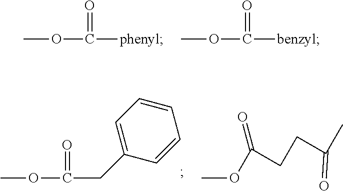

and substituted derivatives thereof. R₃ may be hydrogen or

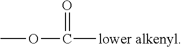

In some embodiments, at least one of R₁ and R₂ are other than hydrogen and R₃ is hydrogen or

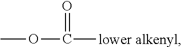

and substituted derivatives thereof. In another embodiment, either R₁ or R₂ is

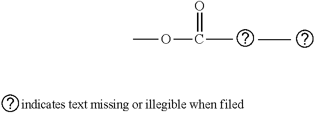

the remaining R₁ or R₂ is a

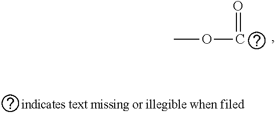

wherein a lower alkyl is between 1 and 7 carbons, and R₃ is hydrogen.

The alkyl, alkenyl, phenyl and benzyl groups of the formulas herein may be unsubstituted or substituted with halogens, preferably, chlorine, fluorine or bromine, nitro, amino, and/or similar type radicals.

In a further embodiment, the invention achieves these objects and satisfies additional objects and advantages by providing novel and surprisingly effective methods and compositions for modulating cell signaling pathways and/or treating diseases or conditions associated with diseases such as Parkinson's disease using an exemplary phorbol ester composition such as 12-O-tetradecanoylphorbol-13-acetate (TPA) of Formula II, below:

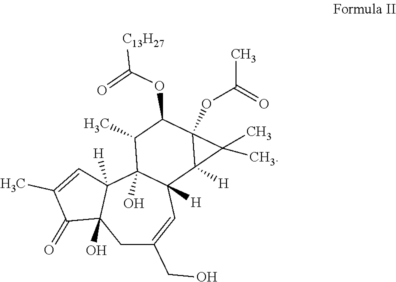

Useful phorbol esters and related compounds and derivatives within the formulations and methods of the invention include, but are not limited to, other pharmaceutically acceptable active salts of said compounds, as well as active isomers, enantiomers, polymorphs, glycosylated derivatives, solvates, hydrates, and/or prodrugs of said compounds. Exemplary forms of phorbol esters for use within the compositions and methods of the invention include, but are not limited to, phorbol 13-butyrate; phorbol 12-decanoate; phorbol 13-decanoate; phorbol 12,13-diacetate; phorbol 13,20-diacetate; phorbol 12,13-dibenzoate; phorbol 12,13-dibutyrate; phorbol 12,13-didecanoate; phorbol 12,13-dihexanoate; phorbol 12,13-dipropionate; phorbol 12-myristate; phorbol 13-myristate; phorbol 12-myristate-13-acetate (also known as TPA or PMA); phorbol 12,13,20-triacetate; 12-deoxyphorbol 13-angelate; 12-deoxyphorbol 13-angelate 20-acetate; 12-deoxyphorbol 13-isobutyrate; 12-deoxyphorbol 13-isobutyrate-20-acetate; 12-deoxyphorbol 13-phenylacetate; 12-deoxyphorbol 13-phenylacetate 20-acetate; 12-deoxyphorbol 13-tetradecanoate; phorbol 12-tigliate 13-decanoate; 12-deoxyphorbol 13-acetate; phorbol 12-acetate; and phorbol 13-acetate.

Mammalian subjects amenable to treatment with phorbol esters of Formula I, particularly TPA, according to the methods of the invention include, but are not limited to individuals with Parkinson's disease. Symptoms of Parkinson's disease that may be treated or prevented by the use of the compositions and methods described herein include, but are not limited to, tremor at rest, stiffness, bradykinesia, rigidity, speech impairment, a feeling of weakness in the limbs, lack of dexterity, poor coordination, imbalance, cognitive impairment, dementia, mood impairment, drowsiness, sleep disturbances, autonomic dysfunction and postural instability. Subjects amenable to treatment with phorbol esters of Formula I, particularly TPA, or derivatives of the phorbol esters of Formula I including pharmaceutically acceptable salts, enantiomers, isomers, polymorphs, prodrugs, solvates and hydrates include those suffering from symptoms of Parkinson's disease or symptoms related to Parkinson's disease such as tremor at rest, stiffness, bradykinesia, rigidity, speech impairment, a feeling of weakness in the limbs, lack of dexterity, poor coordination, imbalance, cognitive impairment, dementia, mood impairment, drowsiness, sleep disturbances, autonomic dysfunction and postural instability.

These and other subjects are effectively treated, prophylactically and/or therapeutically, by administering to the subject an effective amount of a phorbol ester of Formula I sufficient to prevent or treat paralysis, increase spatial awareness, decrease memory loss, decrease aphasia, increase coordination and balance, improve cognition, decrease or eliminate tremors, decrease or eliminate stiffness and rigidity, improve sleep quality, increase stability, improve mobility, improve bladder control, ease muscle or joint aches, improve vision, and/or improve muscle control.

Therapeutically useful methods and formulations of the invention will effectively use a phorbol ester of Formula I in a variety of forms, as noted above, including any active, pharmaceutically acceptable salts of said compounds, as well as active isomers, enantiomers, polymorphs, solvates, hydrates, prodrugs, and/or combinations thereof. TPA of formula II is employed as an illustrative embodiment of the invention within the examples herein below.

Within additional aspects of the invention, combinatorial formulations and methods are provided which employ an effective amount of a phorbol ester of Formula I in combination with one or more secondary or adjunctive active agent(s) that is/are combinatorially formulated or coordinately administered with the phorbol ester compound of Formula I to yield an effective response in the subject.

Exemplary combinatorial formulations and coordinate treatment methods in the treatment of Parkinson's disease employ the phorbol ester compound of Formula I in combination with one or more additional Parkinson's disease treating or other indicated, secondary, or adjunctive therapeutic agents. The secondary or adjunctive therapeutic agents used in combination with a phorbol ester, e.g., TPA, in these embodiments may possess direct or indirect anti-Parkinsonian effects, alone or in combination with, e.g. TPA; may exhibit other useful adjunctive therapeutic activity in combination with, e.g. TPA (dopamine increasing, catechol-O-methyl transferase inhibiting, aromatic L-amino acid decarboxylase inhibiting, dopamine agonist, neuroprotective, anticholinergic); or may exhibit adjunctive therapeutic activity useful for treating or preventing side effects of Parkinson's disease or associated symptoms alone or in combination with, e.g. TPA.

Useful adjunctive or secondary therapeutic agents in these combinatorial formulations and coordinate treatment methods for the prevention or treatment of symptoms of Parkinson's disease in a mammalian subject include, but are not limited to, carbidopa/levodopa, tolcapone, MAO-B inhibitors, pyridoxine, amantadine, selegiline, rasagiline, anticholinergics, cholinesterase inhibitors, COMT Inhibitors, and dopamine agonists including, but not limited to, apomorphine, atropine, benztropine, biperiden, bromocriptine, cabergoline, ciladopa, dihydrexidine, dinapsoline, doxanthrine, entacapone, epicriptine, lisuride, pergolide, piribedil, pramipexole, propylnorapomorphine, quinagolide, rivastigmine, ropinirole, rotigotine, roxindole, sumanirole, trihexphenidyl. In addition, adjunctive or secondary therapies may be used such as, but not limited to, antioxidants, amino acid therapy, deep brain stimulation, diet modification, exercise, herbal supplementation, hormone therapy, mineral supplementation, physical therapy, and/or lesion formation.

The forgoing and additional objects, features, aspects and advantages of the present invention will become apparent from the following detailed description.

### DETAILED DESCRIPTION

Novel methods and compositions have been identified for use in treating chronic or recurring conditions, or to repair the damage left b) episodes of illness or treatment of illness in mammalian subjects, including humans. In various embodiments, the methods and compositions as described herein are effective in treating Parkinson's disease and symptoms caused by or resembling to Parkinson's disease.

In additional embodiments, the methods and compositions described herein are effective to prevent or treat paralysis, increase spatial awareness, decrease memory loss, decrease aphasia, improve coordination and balance, improve cognition, decrease or eliminate tremors, decrease or eliminate stiffness and rigidity, improve sleep quality, increase stability, improve mobility, improve bladder control, increase continence, improve appetite, ease muscle or joint aches, improve vision, and/or improve muscle control.

Formulations and methods provided herein employ a phorbol ester or derivative compound of Formula I, below,

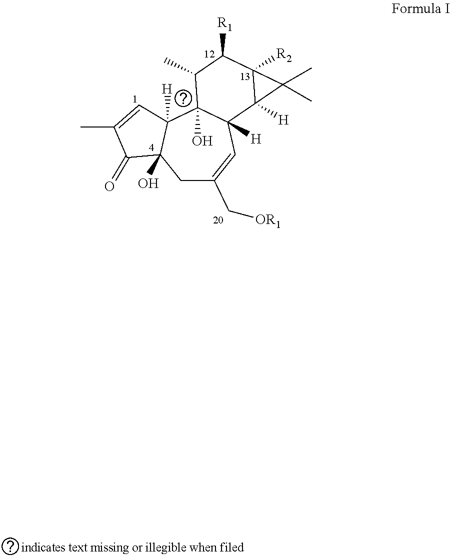

wherein R₁ and R₂ may be hydrogen; hydroxyl;

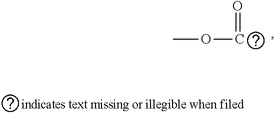

wherein the alkyl group contains 1 to 15 carbon atoms;

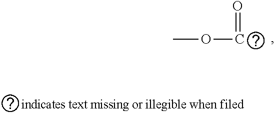

wherein a lower alkenyl group contains between 1 to 7 carbon atoms;

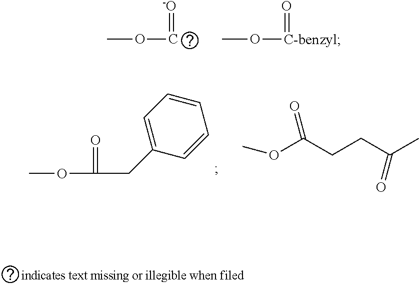

and substituted derivatives thereof. R₃ may be hydrogen or

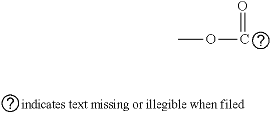

including all active pharmaceutically acceptable compounds of this description as well as various foreseen and readily provided complexes, salts, solvates, isomers, enantiomers, polymorphs and prodrugs of these compounds and combinations thereof as novel Parkinson's Disease treating compounds.

In some embodiments, at least one of R₁ and R₂ are other than hydrogen and R₃ is hydrogen or

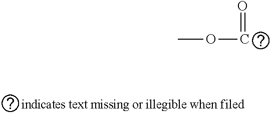

and substituted derivatives thereof. In another embodiment, either R₁ or R₂ is

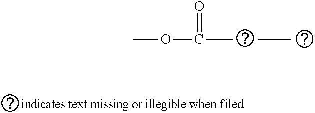

the remaining R₁ or R₂ is a

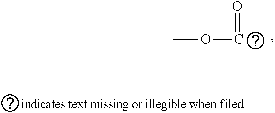

wherein a lower alkyl is between 1 and 7 carbons, and R₃ is hydrogen. The alkyl, alkenyl, phenyl and benzyl groups of the formulas herein may be unsubstituted or substituted with halogens, preferably, chlorine, fluorine or bromine; nitro; amino; and/or similar type radicals. The compounds of Formula I are more fully described in U.S. patent application Ser. No. 12/023,753, filed Jan. 31, 2008, which claims priority benefit of U.S. Provisional patent application Ser. No. 60/898,810, filed Jan. 31, 2007, each of which is incorporated herein in its entirety by reference.

Anti-Parkinsonian formulations and methods provided herein employ a phorbol ester or derivative compound of Formula I, above, including all active pharmaceutically acceptable compounds of this description as well as various foreseen and readily provided complexes, salts, solvates, isomers, enantiomers, polymorphs and prodrugs of these compounds and combinations thereof as anti-Parkinsonian age its.

It is estimated that 7 to 10 million people worldwide suffer from Parkinson's disease, a chronic and progressive neurodegenerative disorder. It is believed to have both genetic and environmental triggers but the exact etiology is not fully understood. Although multiple gene mutations have been identified in Parkinson's disease, only about 10% of all cases are thought to be inherited. Most movement related and autonomic symptoms of Parkinson's disease result from an imbalance between dopamine and acetylcholine. Additionally, low levels of serotonin and norepinephrine contribute to mood changes and depression in the disease. It is also characterized by the presence of Lewy bodies in post mortem observations. The relationship between Parkinson's disease and lewy bodies is not entirely clear but it is believed that lewy bodies may be a product of oxidative stress or perhaps even an immune response to aberrant protein synthesis. Parkinson's disease is characterized by bradykinesia, resting tremor, rigidity, speech impairment, postural instability and dementia.

A broad range of mammalian subjects, including human subjects, are amenable to treatment using the formulations and methods of the invention. These subjects include, but are not limited to, individuals suffering from Parkinson's disease or related conditions or individuals at risk for developing Parkinson's disease or related conditions.

Mammalian subjects amenable to treatment with phorbol esters of Formula I, particularly TPA, according to the methods of the present invention include, but are not limited to, mammalian subjects with Parkinson's disease and/or symptoms related to Parkinson's disease.

Within the methods and compositions of the invention, one or more phorbol ester compound(s) of Formula I as disclosed herein is/are effectively formulated or administered as an agent effective for preventing or treating Parkinson's disease. In exemplary embodiments, TPA is demonstrated for illustrative purposes to be an effective agent in pharmaceutical formulations and therapeutic methods, alone or in combination with one or more adjunctive therapeutic agent(s). The present disclosure further provides additional, pharmaceutically acceptable phorbol ester compounds in the form of a native or synthetic compound, including complexes, derivatives, salts, solvates, isomers, enantiomers, polymorphs, and prodrugs of the compounds disclosed herein, and combinations thereof, which are effective as therapeutic agents within the methods and compositions of the invention in the prevention or treatment of Parkinson's disease and symptoms thereof.

Phorbol is a natural, plant-derived polycyclic alcohol of the tigliane family of diterpenes. It was first isolated in 1934 as the hydrolysis product of Croton oil derived from the seeds of Croton tiglium. It is well soluble in most polar organic solvents and in water. Esters of phorbol have the general structure of Formula I, below:

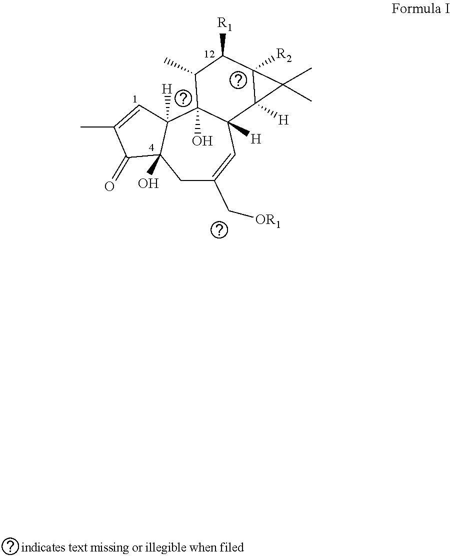

wherein R₁ and R₂ are selected from the group consisting of hydrogen; hydroxyl;

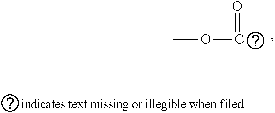

wherein the alkyl group contains 1 to 15 carbon atoms;

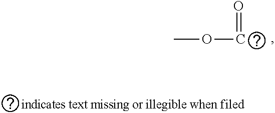

wherein a lower alkenyl group contains between 1 to 7 carbon atoms;

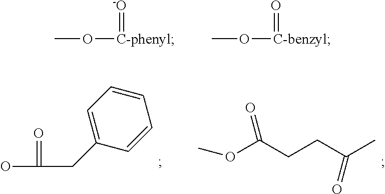

and substituted derivatives thereof and R₃ may be hydrogen,

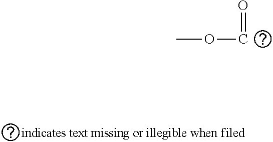

or substituted derivatives thereof as well as pharmaceutically acceptable salts, enantiomers, polymorphs, prodrugs solvates and hydrates of compounds of Formula I and substituted derivatives thereof.

The term “lower alkyl” or “lower alkenyl” as used herein means moieties containing 1-7 carbon atoms. In the compounds of the Formula I, the alkyl or alkenyl groups may be straight or branched chain. In some embodiments, either or both R₁ or R₂, are a long chain carbon moiety (i.e., Formula I is decanoate or myristate).

The alkyl, alkenyl, phenyl and benzyl groups of the formulas herein may be unsubstituted or substituted with halogens, preferably, chlorine, fluorine or bromine; nitro; amino and similar type radicals.

Organic and synthetic forms of phorbol esters, including any preparations or extracts from herbal sources such as Croton tiglium, are contemplated as useful compositions comprising phorbol esters (or phorbol ester analogs, related compounds and/or derivatives) for use within the embodiments herein. Useful phorbol esters and/or related compounds for use within the embodiments herein will typically have a structure as illustrated in Formula I, although functionally equivalent analogs, complexes, conjugates, and derivatives of such compounds will also be appreciated by those skilled in the art as within the scope of the invention.

In more detailed embodiments, illustrative structural modifications according to Formula I above will be selected to provide useful candidate compounds for treating Parkinson's disease, wherein: at least one of R₁ and R₂ are other than hydrogen and R₃ is selected from the group consisting of hydrogen,

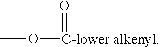

and substituted derivatives thereof. In another embodiment, either R₁ or R₂ is

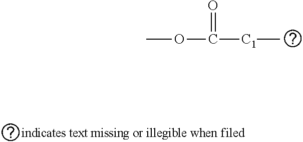

the remaining R₁ or R₂ is

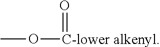

and R₃ is hydrogen.

An exemplary embodiment of a phorbol ester compound of Formula I useful in the treatment of Parkinson's disease, is found in phorbol 12-myristate-13-acetate (also known as PMA or 2-O-tetradecanoyl-phorbol-13-acetate (TPA)) shown in Formula II, below.

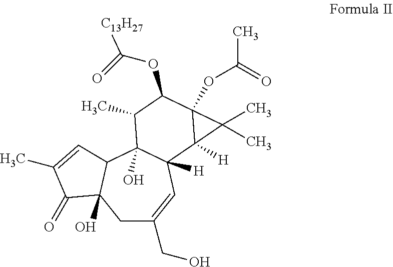

Additional useful phorbol esters and related compounds and derivatives within the formulations and methods of the invention include, but are not limited to, other pharmaceutically acceptable active salts of said compounds, as well as active isomers, enantiomers, polymorphs, glycosylated derivatives, solvates, hydrates, and/or prodrugs of said compounds. Further exemplary forms of phorbol esters for use within the compositions and methods of the invention include, but are not limited to, phorbol 13-butyrate; phorbol 12-decanoate; phorbol 13-decanoate; phorbol 12,13-diacetate; phorbol 13,20-diacetate; phorbol 12,13-dibenzoate; phorbol 12,13-dibutyrate; phorbol 12,13-didecanoate; phorbol 12,13-dihexanoate; phorbol 12,13-dipropionate; phorbol 12-myristate; phorbol 13-myristate; phorbol 12,13,20-triacetate; 12-deoxyphorbol 13-angelate; 12-deoxyphorbol 13-angelate 20-acetate; 12-deoxyphorbol 13-isobutyrate; 12-deoxyphorbol 13-isobutyrate-20-acetate; 12-deoxyphorbol 13-phenylacetate; 12-deoxy phorbol 13-phenylacetate 20-acetate; 12-deoxyphorbol 13-tetradecanoate; phorbol 12-tigliate 13-decanoate; 12-deoxyphorbol 13-acetate; phorbol 12-acetate; and phorbol 13-acetate as shown in Table 1.

TABLE 1
Exemplary
Phorbol Esters

| | |
|---|---|
| Phorbol 13- Butyrate | 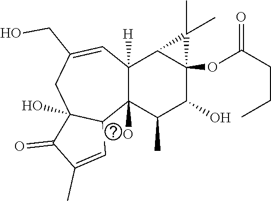 [SMILES: CCCC(=O)O[C@@]12[C@H](O)[C@@H](C)[C@]3(O)C4C=C(C)C(=O)[C@@]4(O)CC(CO)=C[C@H]3[C@@H]1C2(C)C] |
| Phorbol 12- Decanoate | 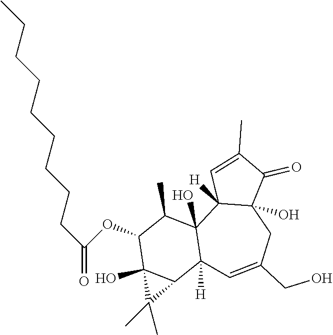 [SMILES: CCCCCCCCCC(=O)O[C@@H]1[C@@H](C)[C@@]2(O)[C@@H](C=C(CO)C[C@]3(O)C(=O)C(C)=C[C@@H]23)[C@@H]2C(C)(C)[C@]12O] |
| Phorbol 13- Decanoate | 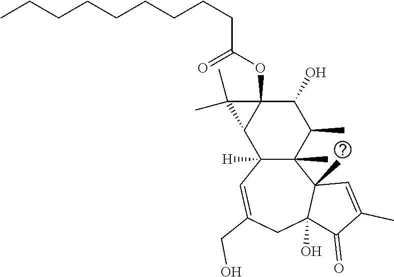 [SMILES: CCCCCCCCCC(=O)O[C@@]12[C@H](O)[C@@H](C)[C@@]3(C)[C@@H](C=C(CO)C[C@]4(O)C(=O)C(C)=C[C@@]34C)[C@@H]1C2(C)C] |
| Phorbol 12,13- Diacetate | 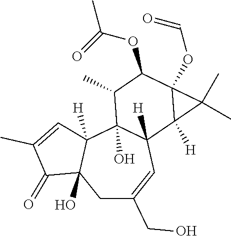 |
| Phorbol 13,20- Diacetate | 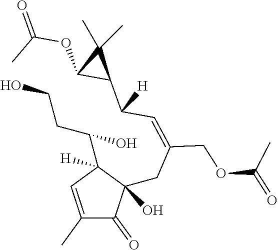 |
| Phorbol 12,13- Dibenzoate | 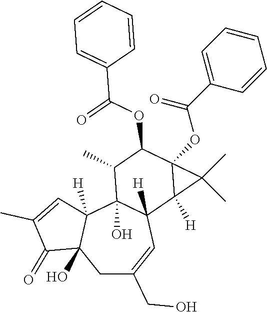 |
| Phorbol 12,13- Dibutyrate | 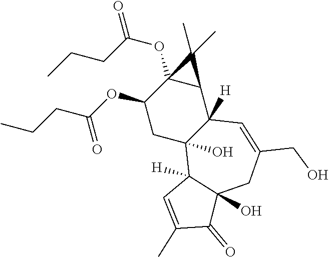 |
| Phorbol 12,13- Didecanoate | 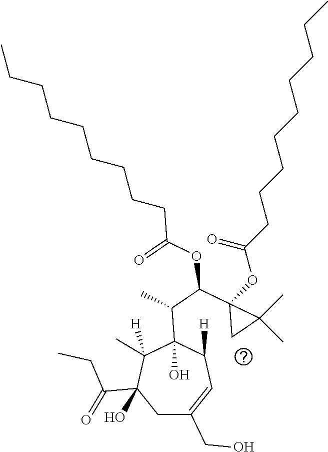 |
| Phorbol 12,13- Dihexanoate | 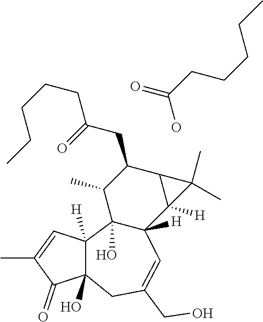 |
| Phorbol 12,13- Dipropionate | 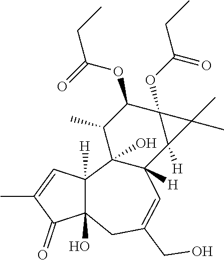 |
| Phorbol 12- Myristate | 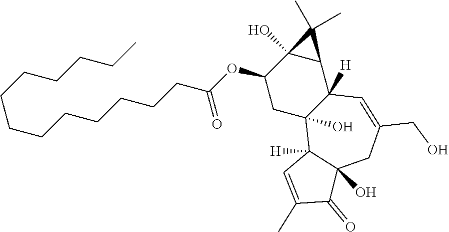 |
| Phorbol 13- Myristate | 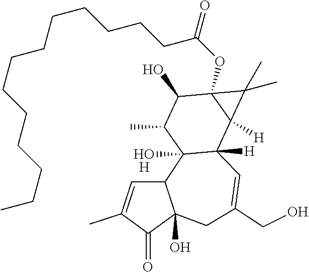 |
| Phorbol 12- Myristate-13- Acetate (also known as TPA or PMA) | 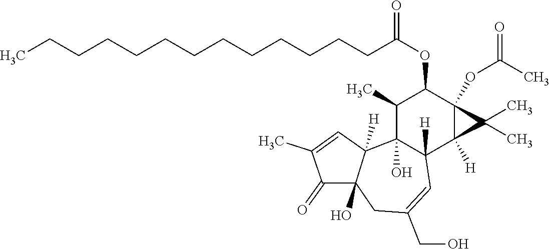 |
| Phorbol 12,13,20- Triacetate | 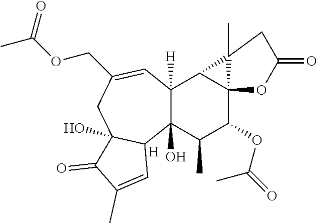 |
| 12-Deoxyphorbol 13-Angelate | 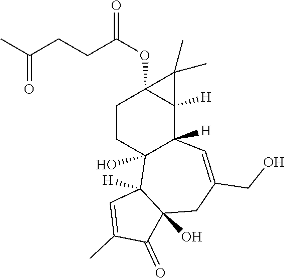 [SMILES: CC(=O)CCC(=O)O[C@@]12CC[C@@]3(O)[C@@H](C=C(CO)C[C@]4(O)C(=O)C(C)=C[C@@H]34)[C@@H]1C2(C)C] |
| 12-Deoxyphorbol 13-Angelate 20- Acetate | 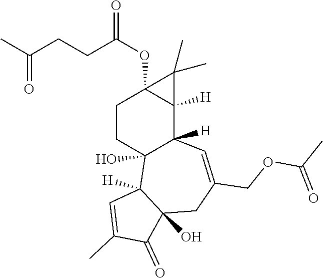 [SMILES: CC(=O)CCC(=O)O[C@@]12CC[C@@]3(O)[C@@H](C=C(COC(C)=O)C[C@]4(O)C(=O)C(C)=C[C@@H]34)[C@@H]1C2(C)C] |
| 12-Deoxyphorbol 13-Isobutyrate | 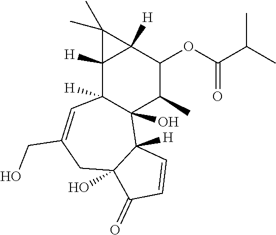 [SMILES: CC(C)C(=O)OC1[C@@H]2[C@H]([C@@H]3C=C(CO)C[C@]4(O)C(=O)C=C[C@H]4[C@@]3(O)[C@@H]1C)C2(C)C] |
| 12-Deoxyphorbol 13-lsobutyrate-20- Acetate | 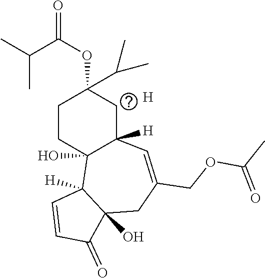 |
| 12-Deoxyphorbol 13-Phenylacetate | 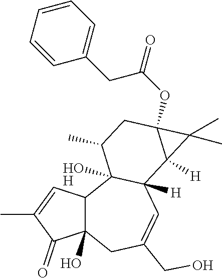 |
| 12-Deoxyphorbol 13-Phenylacetate 20-Acetate | 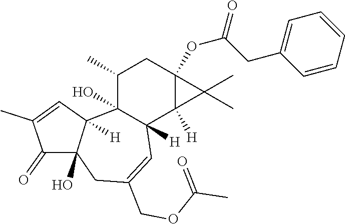 |
| 12-Deoxyphorbol 13- Tetradecanoate | 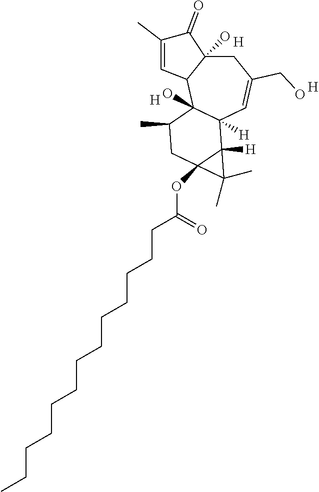 |
| Phorbol 12- Tigliate 13- Decanoate | 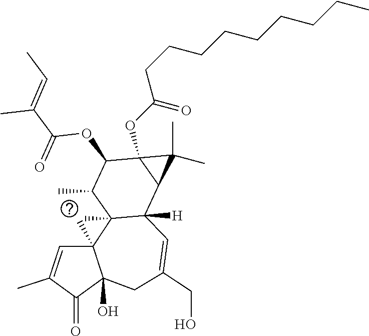 [SMILES: C/C=C(\C)C(=O)O[C@@H]1[C@@H](C)[C@]23C[C@]24C=C(C)C(=O)[C@@]4(O)CC(CO)=C[C@H]3[C@@H]2C(C)(C)[C@]12OC(=O)CCCCCCCCC] |
| 12-Deoxyphorbol 13-Acetate | 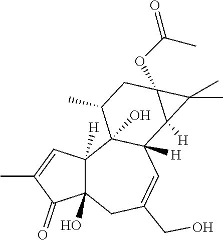 |
| Phorbol 12- Acetate | 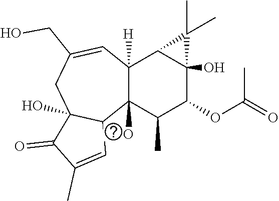 [SMILES: CC(=O)O[C@@H]1[C@@H](C)[C@]2(O)C3C=C(C)C(=O)[C@@]3(O)CC(CO)=C[C@H]2[C@@H]2C(C)(C)[C@]12O] |
| Phorbol 13- Acetate | 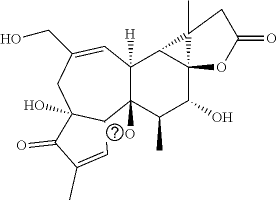 |

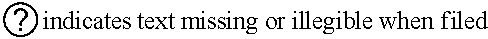

Compositions as described herein further comprise Parkinson's disease treating compositions comprising an effective amount of a phorbol ester compound of Formula I which is effective for prophylaxis and/or treatment of Parkinson's disease or related symptoms in a mammalian subject. A “Parkinson's disease treating,” “dopamine enhancing,” “catechol-O-methyl transferase inhibiting,” “aromatic L-amino acid decarboxylase inhibiting.” “dopamine agonist,” “neuroprotective,” or “anticholinergic” effective amount of the active compound is therapeutically effective in a single or multiple unit dosage form, over a specified period of therapeutic intervention, to measurably alleviate or prevent one or more of the symptoms of Parkinson's disease in the subject. Within exemplary embodiments, the compositions of the invention are effective in treatment methods to prevent or alleviate symptoms of Parkinson's disease in human and other mammalian subjects suffering from or at risk for Parkinson's disease.

Phorbol ester treating, including Parkinson's disease treating, compositions of the invention typically comprise an effective amount or unit dosage of a phorbol ester compound of Formula I, which may be formulated with one or more pharmaceutically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use. Effective amounts of a phorbol ester compound or related or derivative compound of Formula I (e.g., a unit dose comprising an effective concentration/amount of TPA, or of a selected pharmaceutically acceptable salt, isomer, enantiomer, solvate, polymorph and/or prodrug of TPA) will be readily determined by those of ordinary skill in the art, depending on clinical and patient-specific factors. Suitable effective unit dosage amounts of the active compounds for administration to mammalian subjects, including humans, may range from about 10 to about 1500 μg, about 20 to about 1000 μg, about 25 to about 750 μg, about 50 to about 500 μg, about 150 to about 500 μg, about 125 μg to about 500 μg, about 180 to about 500 μg, about 190 to about 500 μg, about 220 to about 500 μg, about 240 to about 500 μg, about 260 to about 500 μg, about 290 to about 500 μg. In certain embodiments, the disease treating effective dosage of a phorbol ester compound or related or derivative compound of Formula I may be selected within narrower ranges of, for example, 10 to 25 μg, 30-50 μg, 75 to 100 μg, 100 to 300 μg, or 150 to 500 μg. These and other effective unit dosage amounts may be administered in a single dose, or in the form of multiple daily, weekly or monthly doses, for example in a dosing regimen comprising from 1 to 5, or 2 to 3, doses administered per day, per week, or per month. In one exemplary embodiment, dosages of 10 to 30 μg, 30 to 50 μg, 50 to 100 μg, 100 to 300 μg, or 300 to 500 μg, are administered one, two, three, four, or five times per day. In more detailed embodiments, dosages of 50-100 μg, 100-300 μg, 300-400 μg, or 400-600 μg are administered once or twice daily. In a further embodiment, dosages of 50-100 μg, 100-300 μg, 300-400 μg, or 400-600 μg are administered every other day. In alternate embodiments, dosages are calculated based on body weight, and may be administered, for example, in amounts from about 0.5 μg/m² to about 300 μg/m² per day, about 1 μg/m² to about 200 μg/m², about 1 μg/m² to about 187.5 μg/ml² per day, about 1 μg/m² per day to about 175 μg/m per day, about 1 μg/ml² per day to about 157 μg/m² per day, about 1 μg/m² to about 125 μg/m² per day, about 1 μg/ml² to about 75 μg/m² per day, 1 μg/m² to about 50 μg/m² per day, 2 μg/ml² to about 50 μg/m per day, 2 μg/m² to about 30 μg/m² per day or 3 μg/m² to about 30 μg/m² per day.

In other embodiments, dosages may be administered less frequently, for example, 0.5 μg/m² to about 300 μg/ml² every other day, about 1 μg/m² to about 200 μg/m², about 1 μg/m² to about 187.5 μg/m² every other day, about 1 μg/m² to about 175 μg/m² every other day, about 1 μg/m² per day to about 157 μg/m² every other day about 1 μg/m² to about 125 μg/m² every other day, about 1 μg/m² to about 75 μg/m² every other day, 1 μg/m² to about 50 μg/m² every other day, 2 μg/m² to about 50 μg/m² every other day, 2 μg/m² to about 30 μg/m² per day or 3 μg/m² to about 30 μg/m² per day. In additional embodiments, dosages may be administered 3 times/week, 4 times/week, 5 times/week, only on weekdays, only in concert with other treatment regimens, on consecutive days, or in any appropriate dosage regimen depending on clinical and patient-specific factors

The amount, timing and mode of delivery of compositions of the invention comprising a Parkinson's disease treating effective amount of a phorbol ester compound of Formula I will be routinely adjusted on an individual basis, depending on such factors as weight, age, gender, and condition of the individual, the acuteness of the cytopathic disease and/or related symptoms, whether the administration is prophylactic or therapeutic, and on the basis of other factors known to effect drug delivery, absorption, pharmacokinetics, including half-life, and efficacy.

An effective dose or multi-dose treatment regimen for the instant disease treating “dopamine enhancing,” “catechol-O-methyl transferase inhibiting,” “aromatic L-amino acid decarboxylase inhibiting,” “dopamine agonist,” “neuroprotective,” “anticholinergic,” “muscle relaxant” formulations of the invention will ordinarily be selected to approximate a minimal dosing regimen that is necessary and sufficient to substantially prevent or alleviate the symptoms of the disease including Parkinson's disease, and/or to substantially prevent or alleviate one or more symptoms associated with Parkinson's disease, in the subject. A dosage and administration protocol will often include repeated dosing therapy over a course of several days or even one or more weeks or years. An effective treatment regime may also involve prophylactic dosage administered on a day or multi-dose per day basis lasting over the course of days, weeks, months or even years.

Effectiveness of the compositions and methods of the invention in the treatment of Parkinson's disease may be demonstrated by a decrease in the symptoms of Parkinson's including, but not limited to tremor, bradykinesia, rigidity, speech impairment, postural instability and dementia. Effectiveness of the phorbol ester compounds of the present invention in the treatment of Parkinson's disease may further be demonstrated by an increase in dopamine and/or norepinephrine levels. Such levels may increase 10%, 20%, 30%, 50% or greater increase, up to a 75-90%, or 95% or greater of normal levels.

Effectiveness of the compositions and methods of the invention in the treatment of Parkinson's disease may be indicated by a decrease in the presence of Lewy bodies and a-synuclein in cases of Parkinson's disease with dementia. Effectiveness may also be demonstrated through the use of animal models, such as MPTP induced Parkinson's, rotenone induced Parkinson's, surgically induced Parkinson's, paraquat induced Parkinson's, 6-OHDA induced Parkinson's, or α-synuclein overexpression in mice. The use of the compositions and methods of the invention will decrease the symptoms of Parkinson's disease expressed in these models by 0%, 20%, 30%, 50% or more, up to a 75-90%, 96% or greater decrease over control animals.

Within additional aspects of the invention, combinatorial disease treating “Parkinson's disease treating,” “dopamine enhancing,” “catechol-O-methyl transferase inhibiting,” “aromatic L-amino acid decarboxylase inhibiting,” “dopamine agonist,” “neuroprotective,” “anticholinergic,” “muscle relaxant” formulations and coordinate administration methods are provided which employ an effective amount of a phorbol ester compound of Formula I and one or more secondary or adjunctive agent(s) that is/are combinatorially formulated or coordinately administered with the phorbol ester compound of Formula I to yield a combined, multi-active disease treating composition or coordinate treatment method. Exemplary combinatorial formulations and coordinate treatment methods in this context employ the phorbol ester of Formula I in combination with the one or more secondary Parkinson's treatment agent(s), or with one or more adjunctive therapeutic agent(s) that is/are useful for treatment or prophylaxis of the targeted (or associated) disease, condition and/or symptom(s) in the selected combinatorial formulation or coordinate treatment regimen. For most combinatorial formulations and coordinate treatment methods of the invention, a phorbol ester compound of Formula I or related or derivative compound is formulated, or coordinately administered, in combination with one or more secondary or adjunctive therapeutic agent(s), to yield a combined formulation or coordinate treatment method that is combinatorially effective or coordinately useful to treat in the prevention or treatment of Parkinson's disease employ the phorbol ester compound of Formula I in combination with one or more additional, neuroprotective or other indicated, secondary or adjunctive therapeutic agents that is/are useful for treatment or prophylaxis of the targeted disease, condition and/or symptom(s). For most combinatorial formulations and coordinate treatment methods of the invention, a phorbol ester compound of Formula I or related or derivative compound is formulated, or coordinately administered, in combination with one or more secondary or adjunctive therapeutic agent(s), to yield a combined formulation or coordinate treatment method that is combinatorially effective or coordinately useful to prevent or treat Parkinson's disease. Exemplary combinatorial formulations and coordinate treatment methods in this context employ a phorbol ester compound of Formula I in combination with one or more secondary or adjunctive therapeutic agents selected from carbidopa/levodopa, tolcapone, MAO-B inhibitors, pyridoxine, amantadine, selegiline, rasagiline, anticholinergics, cholinesterase inhibitors, COMT Inhibitors, and dopamine agonists including, but not limited to, apomorphine, atropine, benztropine, biperiden, bromocriptine, cabergoline, ciladopa, dihydrexidine, dinapsoline, doxanthrine, entacapone, epicriptine, lisuride, pergolide, piribedil, pramipexole, propylnorapomorphine, quinagolide, rivastigmine, ropinirole, rotigotine, roxindole, sumanirole, trihexphenidyl. In addition, adjunctive or secondary therapies may be used such as, but not limited to, antioxidants, amino acid therapy, deep brain stimulation, diet modification, exercise, herbal supplementation, hormone therapy and/or mineral supplementation.

In certain embodiments the invention provides combinatorial disease treating “Parkinson's disease treating,” “dopamine enhancing,” “catechol-O-methyl transferase inhibiting,” “aromatic L-amino acid decarboxylase inhibiting,” “dopamine agonist,” “neuroprotective,” “anticholinergic,” “muscle relaxant” formulations comprising a phorbol ester and one or more adjunctive agent(s) having disease treating activity. Within such combinatorial formulations, a phorbol ester of Formula I and the adjunctive agent(s) having disease treating activity will be present in a combined formulation in disease treating (“Parkinson's disease treating.” “dopamine enhancing,” “catechol-O-methyl transferase inhibiting,” “aromatic L-amino acid decarboxylase inhibiting.” “dopamine agonist,” “neuroprotective,” “anticholinergic,” “muscle relaxant,”) effective amounts, alone or in combination. In exemplary embodiments, a phorbol ester compound of Formula I and a non-phorbol ester agent(s) will each be present in a disease treating/preventing amount (i.e., in singular dosage which will alone elicit a detectable alleviation of symptoms in the subject). Alternatively, the combinatorial formulation may comprise one or both the phorbol ester compound of Formula I and the non-phorbol ester agents in sub-therapeutic singular dosage amount(s), wherein the combinatorial formulation comprising both agents features a combined dosage of both agents that is collectively effective in eliciting a cytopathic disease or condition symptom alleviating response. Thus, one or both of the phorbol ester of Formula I and non-phorbol ester agents may be present in the formulation, or administered in a coordinate administration protocol, at a sub-therapeutic dose, but collectively in the formulation or method they elicit a detectable decrease in symptoms of cytopathic disease in the subject.

To practice coordinate administration methods of the invention, a phorbol ester compound of Formula I may be administered, simultaneously or sequentially, in a coordinate treatment protocol with one or more of the secondary or adjunctive therapeutic agents contemplated herein. Thus, in certain embodiments a compound is administered coordinately with a non-phorbol ester agent, or any other secondary or adjunctive therapeutic agent contemplated herein, using separate formulations or a combinatorial formulation as described above (i.e., comprising both a phorbol ester compound of Formula I or related or derivative compound, and a non-phorbol ester therapeutic agent). This coordinate administration may be done simultaneously or sequentially in either order, and there may be a time period while only one or both (or all) active therapeutic agents individually and/or collectively exert their biological activities.

Within exemplary embodiments, a phorbol ester compound of Formula I will be coordinately administered (simultaneously or sequentially, in combined or separate formulation(s)), with one or more Parkinson's disease treating agents. Such coordinate treatment methods may, for example, follow or be derived from various protocols for the treatment of Parkinson's disease. Coordinate treatment methods may, for example, include a phorbol ester and/or treatments for prevention or treatment of Parkinson's disease. A distinguishing aspect of all such coordinate treatment methods is that the phorbol ester compound of Formula I exerts at least some activity, which yields a favorable clinical response in conjunction with a complementary Parkinson's disease preventing or treating agent, or distinct, clinical response provided by the secondary or adjunctive therapeutic agent. Often, the coordinate administration of the phorbol ester compound of Formula I with the secondary or adjunctive therapeutic agent will yield improved therapeutic or prophylactic results in the subject beyond a therapeutic effect elicited by the phorbol ester compound of Formula I, or the secondary or adjunctive therapeutic agent administered alone. This qualification contemplates both direct effects as well as indirect effects. Within exemplary embodiments, a phorbol ester compound of Formula I will be coordinately administered (simultaneously or sequentially, in combined or separate formulation(s)), with one or more secondary Parkinson's disease treating compounds or other indicated or adjunctive therapeutic agents, e.g. carbidopa/levodopa, tolcapone, MAO-B inhibitors, pyridoxine, amantadine, selegiline, rasagiline, anticholinergics, cholinesterase inhibitors, COMT Inhibitors, and dopamine agonists including, but not limited to, apomorphine, atropine, benztropine, biperiden, bromocriptine, cabergoline, ciladopa, dihydrexidine, dinapsoline, doxanthrine, entacapone, epicriptine, lisuride, pergolide, piribedil, pramipexole, propylnorapomorphine, quinagolide, rivastigmine, ropinirole, rotigotine, roxindole, sumanirole, trihexphenidyl. In addition, adjunctive or secondary therapies may be used such as, but not limited to, antioxidants, amino acid therapy, deep brain stimulation, diet modification, exercise, herbal supplementation, hormone therapy, mineral supplementation, physical therapy, and/or lesion formation.

As noted above, in all of the various embodiments of the invention contemplated herein; the disease treating methods and formulations may employ a phorbol ester compound of Formula I in any of a variety of forms, including any one or combination of the subject compound's pharmaceutically acceptable salts, solvates, isomers, enantiomers, polymorphs, solvates, hydrates, and/or prodrugs. In exemplary embodiments of the invention, TPA is employed within the therapeutic formulations and methods for illustrative purposes.

The pharmaceutical compositions of the present invention may be administered by any means that achieve their intended therapeutic or prophylactic purpose. Suitable routes of administration for the compositions of the invention include, but are not limited to, conventional delivery routes, devices and methods including injectable methods such as, but not limited to, intravenous, intramuscular, intraperitoneal, intraspinal, intrathecal, intracerebroventricular, intraarterial, subcutaneous and intranasal routes.

The compositions of the present invention may further include a pharmaceutically acceptable carrier appropriate for the particular mode of administration be ng employed. Dosage forms of the compositions of the present invention include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

If desired, the compositions of the invention can be administered in a controlled release form by use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps or other biocompatible matrices such as cholesterol.

Some phorbol ester compositions of Formula I of the invention are designed for parenteral administration, e.g. to be administered intravenously, intramuscularly, subcutaneously or intraperitoneally, including aqueous and non-aqueous sterile injectable solutions which, like many other contemplated compositions of the invention, may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers. Additional compositions and formulations of the invention may include polymers for extended release following parenteral administration. The parenteral preparations may be solutions, dispersions or emulsions suitable for such administration. The subject agents may also be formulated into polymers for extended release following parenteral administration. Pharmaceutically acceptable formulations and ingredients will typically be sterile or readily sterilizable, biologically inert, and easily administered. Such polymeric materials are well known to those of ordinary skill in the pharmaceutical compounding arts. Parenteral preparations typically contain buffering agents and preservatives, and injectable fluids that are pharmaceutically and physiologically acceptable such as water, physiological saline, balanced salt solutions, aqueous dextrose, glycerol or the like. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In more detailed embodiments, compositions of the invention may comprise a phorbol ester compound of Formula I encapsulated for delivery in microcapsules, microparticles, or microspheres, prepared, for example, by coacervation techniques or by interfacial polymerization, for example, hydroxymethylcellulose or gelatin-microcapsules and poly(methylmethacrylate) microcapsules, respectively; in colloidal drug delivery systems (for example, liposomes, albumin microspheres, microemulsions, nano-particles and nanocapsules); or within macroemulsions.

As noted above, in certain embodiments the methods and compositions of the invention may employ pharmaceutically acceptable salts, e.g., acid addition or base salts of the above-described phorbol ester compounds of Formula I and/or related or derivative compounds. Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like; organic acid salts such as acetate, citrate, lactate, succinate, tartrate, maleate, fumarate, mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparaginate, glutamate, tartrate, and gluconate salts. Suitable base salts are formed from bases that form non-toxic salts, for example aluminum, calcium, lithium, magnesium, potassium, sodium, zinc and diethanolamine salts.

Other detailed embodiments, the methods and compositions of the invention for employ prodrugs of phorbol esters of Formula I. Prodrugs are considered to be any covalently bonded carriers which release the active parent drug in vivo. Examples of prodrugs useful within the invention include esters or amides with hydroxyalkyl or aminoalkyl as a substituent, and these may be prepared by reacting such compounds as described above with anhydrides such as succinic anhydride.

The invention disclosed herein will also be understood to encompass methods and compositions comprising phorbol esters of Formula I using in vivo metabolic products of the said compounds (either generated in vivo after administration of the subject precursor compound, or directly administered in the form of the metabolic product itself). Such products may result for example from the oxidation, reduction, hydrolysis, amidation, esterification and the like of the administered compound, primarily due to enzymatic processes. Accordingly, the invention includes methods and compositions of the invention employing compounds produced by a process comprising contacting a phorbol ester compound of Formula I with a mammalian subject for a period of time sufficient to yield a metabolic product thereof. Such products typically are identified by preparing a radiolabelled compound of the invention, administering it parenterally in a detectable dose to an animal such as rat, mouse, guinea pig, monkey, or to man, allowing sufficient time for metabolism to occur and isolating its conversion products from the urine, blood or other biological samples.

The invention disclosed herein will also be understood to encompass diagnostic compositions for diagnosing the risk level, presence, severity, or treatment indicia of, or otherwise managing diseases including, but not limited to, Parkinson's disease or a related disease or condition in a mammalian subject, comprising contacting a labeled (e.g., isotopically labeled, fluorescent labeled or otherwise labeled to permit detection of the labeled compound using conventional methods) phorbol ester compound of Formula I to a mammalian subject (e.g., to a cell, tissue, organ, or individual) at risk or presenting with one or more symptom(s) of Parkinson's disease, and thereafter detecting the presence, location, metabolism, and/or binding state of the labeled compound using any of a broad array of known assays and labeling/detection methods. In exemplary embodiments, a phorbol ester compound of Formula I is isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. The isotopically-labeled compound is then administered to an individual or other subject and subsequently detected as described above, yielding useful diagnostic and/or therapeutic management data, according to conventional techniques.

### EXAMPLES

The experiments described below demonstrate novel and powerful uses for phorbol esters and related derivative compounds in the treatment of Parkinson's disease and symptoms of Parkinson's disease. These and additional findings are further expanded and elucidated within the following examples.

### Example I Dose

### Ranging Study

Due to the strong local irritation caused by TPA application, TPA was given to patients by intravenous (i.v.) infusion. TPA solution in a sterile syringe was injected into 200 ml of sterile saline and mixed well for i.v. infusion.

### The Toxicity and Side Effects of Different TPA Doses Administered Clinically:

(1) TPA Given at 1 Mg/Patient/Week:

One mg TPA in solution was mixed well with 200 ml of sterile saline for intravenous infusion which was completed in 1 h at the rate of 16 μg/min. One hour after TPA administration, patients started to have chills which lasted for about 30 min, followed by fever, (the patients' temperature reached 37.5-39.5° C. which lasted for 3-5 h, then returned to normal) with light to heavy perspiration. The above symptoms could be alleviated by giving the patients glucocorticoids. TPA at this dose caused a minority of patients to bleed, several patients suffered for a short period of time difficulty in breathing, and Hb was detected in the urine. However, these side effects were short lived and reversible. The cardiac, hepatic, renal and pulmonary functions were all found to be normal.

(2) TPA Given at 0.5 mg/Patient×2/Week: (Two Doses a Week)

0.5 mg of TPA in solution was mixed well with 200 ml of saline for intravenous infusion which was completed in 1 h at the rate of 8 μg/min. The reactions after administration were similar to that of the 1 mg TPA dosage, but to a lesser extent than the 1 mg dose. The patients tolerated the lower dose more easily. Occasionally, Hb was detected in patients' urine. Difficulty in breathing was not observed. The cardiac, hepatic, renal and pulmonary functions were all normal.

(3) TPA Given at 0.25 mg/Patient×4/week:

0.25 mg of TPA in solution was mixed well with 200 ml of saline for intravenous infusion which was completed in 1 h at the rate of 4 μg/min. After administration, symptoms such as chills and fever were also observed, but to a much lesser extent than with the higher dosages. No Hb was detected in the urine, and no patient suffered difficulty in breathing. The cardiac, hepatic, renal and pulmonary functions were all normal.

### Example II

### Effectiveness of TPA in the Treatment of Parkinson's Disease Using a 6-OHDA Model

The action of TPA is investigated using the 6-hydroxydopamine (6-OHDA)-lesioned rat model of Parkinson's disease where the animals have a partial, unilateral loss of dopaminergic neurons induced by a single injection of 6-OHDA in the median forebrain bundle. Upon amphetamine stimulation, the lesioned animals will display ipsiversive rotational behavior due to the imbalance in dopamine release resulting from the unilateral loss of dopaminergic cells. This allows the functional effects of a compound tested in this model to be quantified by counting the number of amphetamine-induced rotations (Ungerstedt, U., 1970). Animals with complete lesion will exhibit contraversive rotational behavior after apomorphine stimulation, making it possible to exclude any fully lesioned animals from the study.

Male Sprague-Dawley rats weighing 280-320 g are housed in a temperature-controlled room under a 12 h light/dark cycle with access to food and water ad libitum. Thirty minutes prior to surgery, animals are injected intraperitoneally with pargyline (5 mg/kg) and desipramine (25 mg/kg). Rats are then placed in a stereotactic frame under general anesthesia and a small bur-hole is made in the right side of the skull. Each animal is given a unilateral injection of 4 μg, 6-OHDA (in 2 μl sterile water with 0.1% ascorbic acid) over 5 minutes into the right medial forebrain bundle at co-ordinates −2.8 mm from bregma, 2 mm lateral to the midline, and 8.6 mm below skull and the rats are then given 5 weeks to recover. The rats are each given 0.05 mg/kg s.c. apomorhine and 5 mg/kg i.p. amphetamine and assessed for partial or complete lesions. The animals with partial lesions as described above are then randomly divided into two groups and injected with either placebo or 0.125 mg/m² of TPA once per day for six days. After six days they are then injected again with 0.05 mg/kg s.c. apomorphine and 5 mg/kg i.p. amphetamine and rotational activity is measured by counting the total number of turns in the 60 minutes following apomorphine or amphetamine administration. The rats are then left alone for two weeks and sacrificed. The brains of the rats are then fixed and immunostained for the tyrosine hydroxylase, and the dopaminergic neurons and their fibers quantified stereologically.

### Example III

### Therapeutic Effect of TPA in the MPTP Model of Parkinson's Disease

The neurotoxin I-methyl-4-phenyl-1,2,3,6-tetrahydropyridine (MPTP) is a complex I (NADH dehydrogenase) mitochondrial respiratory chain inhibitor that is used to induce dopaminergic cell loss (Varastet et al., 1994). This toxin is currently widely used as an animal model for PD (Bezard et al., 1997).

Male C57/BL6 mice of 6-9 months old weighing 30-40 g are divided into two groups. All mice are given MPTP (30 mg/kg i.p.) b.i.d. for 6 days. Mice are injected with 0.125 mg/m² TPA or vehicle 2 hours prior to toxin administration until the day before sacrifice. Eight days after stopping injection of MPTP, the mice are sacrificed by CO₂ and the striata from both sides are dissected out on cold surface and frozen on dry ice. The dopaminergic neuronal survival is assessed by striatal dopamine (DA) content. The dopamine content is assayed by a radioenzymatic method under GLP conditions, but DA can also be measured using high pressure liquid chromatography with electrochemical detection as previously described (Friedemann & Gerhardt 1992).

### Example IV

### Therapeutic Effect of TPA in the Paraquat Model of Parkinson's Disease

Experimental evidence demonstrates that the herbicide Paraquat is involved in the pathogenesis of Parkinson's Disease. Both microinfusion of Paraquat into the Substantia Nigra of the animal and systemic or intraperitoneal treatment lead to selective degeneration of dopaminergic neurons accompanied by behavioral and neuropathological signs of severe non-selective neurotoxicity (Brooks A. I. et al., 1999; Thiruchelvam M. et al., 2002; McCornack A. L. et al., 2002).

In this experiment, male Sprague Dawley rats (210-220 g weight) are kept on a standard laboratory diet for three weeks (relative humidity 50%±10%, temperature 22° C.±1 and in a light/darkness cycle of 12 hours/12 hours) prior to the tests with food and water available ad libitum.

Prior to treatment, the rats are anaesthetized by intraperitoneal injection of 380 mg/kg weight of chloral hydrate; a stainless steel guide cannula (size 25) is implanted unilaterally in the Substantia Nigra with stereotaxic guiding and is secured to the cranium with denial acrylic material.

The animals (20 per test group) are allowed a recovery period of seven days prior to treatment; during this period no change in motor activity or posture is observed. Microinfusions are performed by means of a 10 μl Hamilton syringe connected to an injection cannula by means of a Teflon tube. The Paraquat, TPA (0.125 mg/m²)+Paraquat or the vehicle (0.8% NaCl) are injected at a total volume of 1 μl/minute. The animals are then observed for motor activity and general well-being.

### Example V

### Therapeutic Effect of TPA in the MPTP Model of Parkinson's Disease

MPTP (I-methyl-4-phenyl-1,2,3,6-tetrahydropyridine) is a neurotoxin precursor to MPP+, which causes permanent symptoms of Parkinson's disease by destroying dopaminergic neurons in the substantia nigra of the brain.

C57BL/6J mice are pretreated with either vehicle (1% methyl cellulose) or TPA 30 min prior to administration of MPTP. MPTP is dissolved in isotonic saline (0.9%) and given subcutaneously as a single dose of 15 mg free base/kg body weight to produce a reduction in striatal dopamine to about 0.5 nanomoles/mg protein. Groups of mice (n=8-10 per group) receive either vehicle plus saline, vehicle plus MPTP, or TPA plus MPTP. Seventy two hours after receiving MPTP, mice are sacrificed using cervical dislocation and the striata are excised. The tissue is homogenized in 0.4 N perchloric acid, centrifuged, and the supernatant analyzed by high performance liquid chromatography/electro-chemical detection (HPLC/ED) for dopamine levels. Supernatants are stored in a −90° C. freezer between the time of collection and analysis. TPA is combined with methyl cellulose and homogenized in water for dosing.

Additional studies are carried out to determine the long-term effect on dopamine depletion of TPA. Using the general test method described above, C57BL/6J mice are pretreated with either vehicle or TPA and MPTP and then sacrificed either 3 or 14 days after dosing.

### Example VI

### Effect of TFA on Reserpine and Haloperidol Model of Parkinson's Disease

Reserpine (usual dose 4-5 mg·kg⁻¹ s.c.) works by inhibiting the vesicular monoamine transporter, VMAT2. This leads to loss of storage capacity and hence depletion of brain (and peripheral) monoamines including noradrenaline and 5-HT as well as dopamine. Behaviorally, reserpine induces features of akinesia and hind limb rigidity in rats that are representative of symptoms associated with PD.

Haloperidol works by antagonizing dopamine D₂ and, to a lesser extent, D₁ receptors in medium spiny neurons that comprise the indirect and direct pathways of the motor circuit respectively. The resultant block of striatal dopamine transmission results in abnormal downstream firing within the basal ganglia circuits that is manifest as symptoms of muscle rigidity and catalepsy within 60 min of haloperidol (0.5-5 mg·kg⁻¹ i.p.) injection (see, e.g., Sanberg P R. Haloperidol-induced catalepsy is mediated by postsynaptic dopamine receptors. Nature. 1980; 284:472-473).

Animals are administered injections of dextrose (vehicle), reserpine, or haloperidol. Eighteen hours after reserpine is administered, 0.125 mg/m² TPA is administered. Fifteen minutes after haloperidol is administered, 0.125 mg/m TPA is administered.

Animals are monitored over a 60 min period in all instances in transparent shoebox cages that measure 45×25×20 cm, with a 1-cm depth of wood chips on the cage floor and a plastic grid on top of the cage. Rectangular photocell monitors with a bank of 3 photocell beams surround each test cage. Ambulations (locomotor activity: total number of beam breaks in 1 hour) are recorded by computer and stored for each test session. All data are expressed as total counts over the entire 1 h test period.

### Example VII

### Therapeutic Effectiveness of TPA on Rotenone Model of Parkinson's Disease

The insecticide rotenone is highly lipophilic, readily crossing the blood-brain barrier and diffusing into neurons where, it accumulates within mitochondria and inhibits complex I. The production of ROS, subsequent to glutathione depletion, is thought to induce oxidative stress (Sherer T B, Betarbet R, Testa C M, Seo B B. Richardson J R, Kim J H, et al. Mechanism of toxicity in rotenone models of Parkinson's disease. J Neurosci. 2003: 23:10756-10764.). Oxidative damage, in the form of protein carbonyl formation, has certainly been found in the midbrain, olfactory bulb, striatum and cortex of rats treated with rotenone (Sherer T B, Betarbet R, Testa C M, Seo B B, Richardson J R, Kim J H, et al. Mechanism of toxicity in rotenone models of Parkinson's disease. J Neurosci. 2003; 23:10756-10764.), just as is reported in the PD brain at post-mortem (Alam Z I, Daniel S E, Lees A J, Marsden D C, Jenner P. Halliwell B. A generalised increase in protein carbonyls in the brain in Parkinson's but not incidental Lewy body disease. J Neurochem. 1997; 69:1326-1329).

Forty four male Sprague-Dawley rats weighing 239±5 g (range: 232-249 g) at the start of testing are evaluated for baseline performance in the Corridor, Stepping and Whisker Tests was established over 1 week pre-operatively.

For the Corridor test, hungry rats (deprived of food overnight) are placed into a long, narrow alleyway (150 cm) and are free to retrieve treats from pots on the left and right sides. Trials are completed when rats made a total of 20 retrievals or after a maximum trial time of 5 min has elapsed. Data are expressed as the number of retrievals made from the ipsilateral side as a percentage of the total number of retrievals made. (Dowd E, Monville C, Torres E M, Dunnett S B (2005) The corridor task: a simple test of lateralised response selection sensitive to unilateral dopamine deafferentation and graft-derived dopamine replacement in the striatum. Brain Res Bull 68(1-2):24-30)

For the Stepping Test both hindlimbs and one forelimb are gently restrained by the experimenter, and the number of forehand and backhand adjusting steps made by the unrestrained forelimb is counted when the rat is moved sideways along a table surface for 90 cm over 5 s. (Olsson M, Nikkhah G, Bentlage C, Bjorklund A (1995) Forelimb akinesia in the rat Parkinson model: differential effects of dopamineagonists and nigral transplants as assessed by a new stepping test. J Neurosci 15(5 Pt 2):3863-3875.)

For the Whisker Test of sensorimotor integration both hind limbs and one forelimb are gently restrained by the experimenter, and the number of vibrissac-evoked forelimb-placings made by the unrestrained forelimb is counted when the rats whiskers are brushed against the side of a table. This is repeated 10 times for each rat on both sides. (Schallert T, Fleming S M, Leasure J L, Tillerson J L, Bland S T (2000) CNS plasticity and assessment of forelimb sensorimotor outcome in unilateral rat models of stroke, cortical ablation, parkinsonism and spinal cord injury. Neuropharmacology 39(5):777-787

After evaluation, rats are divided into performance-matched groups and receive rotenone, control infusions, TPA+rotenone, or TPA+control into the striatum at four points along its rostrocaudal axis. Unilateral lesion surgery is conducted under isofluorane anesthesia (5% in 02 for induction and 2% in 02 for maintenance) in a stereotaxic frame with the nose bar set at 2.3 mm. Rotenone (Sigma, Ireland) is dissolved in a solution of DMSO (Sigma, Ireland). Cremophor® (Sigma, Ireland) and saline (1:1:18). The striatum is lesioned by infusion (1μ/min for 3 min with 2 min for diffusion) at four points along its rostrocaudal axis at stereotaxic coordinates AP+1.3, ML±2.7; AP+0.4, ML±3.1; AP −0.4 ML±4.3; AP −1.3, ML±4.7 (from bregma) and DV −5.0 below dura. All rats are lesioned on the side opposite their preferred side as indicated by pre-operative testing in the Corridor Test.

Post-operative behavioral testing is carried out over a 5 week period after which the rats are sacrificed by transcardial fixation and used for quantitative tyrosine hydroxylase and α-synuclein immunohistochemistry. A separate cohort of rats are administered the same rotenone or control infusions and are sacrificed by decapitation 5 weeks after surgery for HPLC analyses.

Rats are sacrificed by terminal anesthesia (50 mg/kg pentobarbital i.p.) and trans-cardially perfused with 100 ml heparinized (5000 U/L, Sigma, Ireland) saline followed by 150 ml 4% paraformaldehyde. Brains are rapidly removed, post-fixed in 4% para-formaldehyde for 4 h and stored in a 25% sucrose plus 0.1% sodium azide solution. Serial coronal sections (40 μm) are cut using a freezing sledge microtome and a 1:6 series of sections is used for all quantitative immunohistochemistry. For immunohistochemical analyses, following quenching of endogenous peroxidase activity (using a solution of 3% hydrogen peroxide/10% methanol in distilled water) and blocking of non-specific secondary antibody binding (using 3% normal horse serum in Tris-buffered saline (TBS) with 0.2% Triton X-100 at room temperature for 1 h), sections are incubated overnight at room temperature with the appropriate primary antibody diluted in TBS with 0.2% Triton X-100 (mouse anti-tyrosine hydroxylase (MAB318, 1:1000. Millipore. UK) and mouse anti-α-synuclein (AB 1903, 1:1000. Abcam, UK)). Sections are then incubated in a biotinylated secondary antibody for 3 h (horse anti-mouse (1:200, Vector, UK)) followed by 2 h incubation in streptavidin-biotin-horseradish peroxidase solution (Vector, UK). Sections are developed in 0.5% solution of diaminobenzidine tetrahydrochloride (Sigma, Ireland) in Tris buffer containing 0.3 μl/ml of hydrogen peroxide. Sections are mounted on gelatin coated microscope slides, dehydrated in ascending concentrations of alcohols, cleared in xylene and cover slipped using DPX mountant (BDH chemicals. UK). Photomicrographs of immunostained sections are captured under bright field illumination and all image analysis is completed using Image J software. For quantification of any rotenone-induced nigro striatal degeneration, tyrosine hydroxylase immunopositive cell bodies in the ipsilateral and contralateral substantia nigra are counted in three coronal sections, and the optical density of tyrosine hydroxylase immunoreactivity in the ipsilateral and contralateral striata are quantified in three coronal sections. For quantification of any rotenone-induced α-synuclein expression, α-synuclein immunopositive cell bodies in the ipsilateral and contralateral substantia nigra are counted in a single coronal section, and the optical density of α-synuclein immunoreactivity in the ipsilateral and contralateral striata are quantified in a single coronal section. To quantify any rotenone-induced striatal damage, ipsilateral and contralateral striatal volume is determined from three coronal sections through the striatum. All quantitative immunohistochemical data on the lesioned side is expressed as a percentage of the intact side.

### HPLC Analyses

Striatal concentrations of dopamine, noradrenaline and 5-HT in the intact and lesioned striata are determined using HPLC with electrochemical detection. Striatal tissue samples are briefly sonicated in 1 ml of mobile phase (0.1 M citric acid, 0.1 M sodium dihydrogenphosphate, 0.1 mM EDTA, 1.4 mM octane-1-sulfonic acid, 10% (v/v) methanol in distilled water, pH 2.8) containing the N-methyl5-hydroxytryptamine (2 ng/20 μl) as the internal standard. Homogenates are centrifuged at 15,000×g for 15 min and 20 μl of the resultant supernatant is injected onto a reverse-phase column (LI Chrosorb RP-18, 25 cm×4 mm internal diameter, particle size 5 μm) for separation of catecholamines (flow rate 1 ml/min). Neurotransmitter concentrations are quantified by electrochemical detection (Shimadzu) and chromatograms are generated using a Merck-Hitachi D-2000 integrator. Striatal concentrations of GABA are determined using reverse-phase I IPLC with fluorometric detection as described previously described (Rea K, Cremers T I, Westerink B H (2005) HPL Conditions are critical for the detection of GABA by microdialysis. J Neurochem 94(3):672-679). In brief, tissue is sonicated and centrifuged as described above and GABA concentrations in the supernatant are determined off-line by pre-column derivatization with o-phthaldialdehyde/mercaptoethanol reagent (50 mg o-phthaldialdehyde (Sigma) dissolved in 1 ml methanol and added to 99 ml 0.5 mol/L NaHCO₃ (pH 9.5) containing 10 μl 2-mercaptoethanol), and separation by reverse-phase HPLC with fluorometric detection. Samples (50 μl) are derivatized with 30 μl o-phthaldialdehyde/mercaptoethanol reagent, mixed and allowed to react for 2 min. Then 50 μl of the reaction mixture is injected by a Gilson401C auto sampler (Gilson, Villiers le Bel, France) onto the HPLC apparatus. The derivatization mixture is separated using anisocratic mobile phase and measured by fluorometric detection. The mobile phase consisted of 70 mm di-sodium hydrogen phosphate, 400 μm EDTA, 0.15% (v/v) tetrahydrofurane and methanol (30% (v/v)). The pH of the mobile phase is adjusted to 5.26 with phosphoric acid. The HPLC system consisted of a SupercosilLC-18-DB column (150×4.6 mm, particle size 3 m; Supelco Inc., Bella Fonte, Pa., USA), a Gynkotec 300C high precision pump (flow rate 0.95, or 1.00 ml/min; Gynkotec, Germering, Germany) and a JASCO FP-1520 fluorometric detector (excitation λ-350 nm, emission λ-450 nm; Jasco Corporation, Tokyo, Japan). (Connor T J, Kelly J P, Leonard B E (1997) Forced swim test-induced neurochemical endocrine, and immune changes in the rat. Pharmacol Biochem Behav 58(4):961-967.)

### Statistical Analyses

Data are analyzed using a Student's t-test or ANOVA (one-way or two-way with repeated measures followed by post-hoc Newman-Keuls) where appropriate. All tests used are indicated at the relevant point in the text. Differences between groups are considered statistically significant when P<0.05. All data are expressed as mean f SEM.

### Example VIII

### Effectiveness of Phorbol Esters as Catechol O-methyltransferase Inhibitors

The actively of the phorbol esters as catechol O-methyltransferase (COMT) inhibitors may be readily determined without undue experimentation using a fluorescence or fluorescence polarization (FP) methodology that is well known in the art (Kurkela M et al., Anal Biochem (331) 2004, 198-200 and Graves, T L et al. Anal Biochem (373) 2008, 296-306). Any compound exhibiting an IC50 below 1 μM would be considered a COMT inhibitor as defined herein.

### Assay 1

Recombinant human S-COMT (12 nM) is preincubated with the phorbol ester and 400 μM S-adenosyl-L-methionine in 100 mM Na₂HPO₄ buffer, pH 7.4, containing 5 mM MgCl₂ for 60 min at 37° C. The reaction is started with the addition of the substrate esculetir for a final concentration of 2 μM and the production of O-methylated esculetin is followed with the FlexStation fluorescence plate reader (Molecular Probes, USA) using excitation at 355 nm and emission at 460 nm. The inhibitor dissociation constant, K, of the studied compounds is calculated using the Morrison equation, which takes the tight binding inhibition into account (Copeland, R. A. Evaluation of Enzyme Inhibitors in Drug Discovery: A Guide for Medicinal Chemists and Pharmacologists, John Wiley & Sons, Inc., Hoboken, N.J., 2005, pp. 185-187): v, _ₜ (E+I+Kᵢ)−V(E+I+Kᵢ)²−4E−I wherein v₀ and v; are the reaction velocities in the absence and presence, respectively, of the inhibitor, E is the active enzyme concentration, and I is the inhibitor concentration. The data are analyzed with GraphPad Prism version 4.00 software (GraphPad Software, San Diego, Calif., USA).

### Assay 2

Recombinant human S-COMT (0.8 nM) is incubated with the phorbol ester and 200 μM S-adenosyl-L-methionine in 100 mM Na₂HPO₄ buffer, pH 7.4, containing 5 mM MgCl₂ for 30 min at 37° C. The reaction is started with the addition of esculetin for a final concentration of 0.5 μM and the reaction mixture with total volume of 200 μl is incubated for 30 min at 37° C. The reaction is stopped with 20 μl of 4 M HClO₄ and the precipitated protein is removed by Sirocco protein precipitation plate (centrifuged at 4° C. for 10 min at 3000 g). O-Methylated esculetin is detected by Waters HT Alliance HPLC setup with Waters 474 fluorescence detector (Ex 460 nm, Em 460 nm, Gain 100). The analytes are separated isocratically using 0.1 M Na₂HPO₄, 20 mM citric acid, 0.15 mM EDTA, pH 3.2, in 40% methanol as mobile phase and Waters Spherisorb ODS2 (3μηι, 4.6 mm×100 mm) column. O-Methylated esculetin concentrations are calculated based on the standard curve and the ¾ values are calculated using the Morrison equation as in Assay I.

### Example IX

### Effectiveness of Phorbol Esters as Decarboxylase Inhibitors

Phorbol esters are assayed for the inhibition of mammalian decarboxylase based on the formation of serotonin. Serotonin decreases generation of dopaminergic neurons from mesencephalic precursors via serotonin type 7 and type 4 receptors (Parga, J., Rodriguez-Pallares, J., Muñoz, A., Guerra, M. J. and Labandeira-Garcia, J. L. (2007), Serotonin decreases generation of dopaminergic neurons from mesencephalic precursors via serotonin type 7 and type 4 receptors. Devel Neurobio, 67: 10-22. doi: 10.1002/dneu.20306). Groups of mice are pretreated 16 hours in advance with an MAO-B inhibitor are given a combined dose of the phorbol ester being tested and 100 mg/kg i.p. of 5-hydroxytryptophane. Oral doses are administered separately 30 minutes prior to the 5-hydroxytryptophane. Forty-five minutes after administration of the 5-hydroxytryptophane, the mice are sacrificed. The kidneys of each group are pooled, homogenized in water assayed for serotonin. The results are expressed in terms of percent inhibition of serotonin in the kidneys as compared to a control group similarly treated but not given a protease ester.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited with the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

Abrahm J. L., Gerson S. L., Hoxie J. A., Tannenbaum s. h., Cassileth p. A., Cooper R. A. Differential effects of phorbol esters on normal myeloid precursors and leukemic cells. Cancer Res. 46, 3711-3716 (1986).
Ando 1., Crawfor D. H. et al. Phorbol ester-induced expression and function of the interleukin 2 receptor in human B lymphocytes. Eur J Immunol. 15(4), 341-4 (1985).
Aye M. T., Dunrne J. V. Opposing effects of 12-O-tetradecanoylphorbol 13-acetate on human myeloid and lymphoid cell proliferation. J Cell Physiol. 114(2), 209-14 (1983).
Bauer I., Al Sarraj J. et al. Interleukin-I beta and tetradecanoylphorbol acetate-induced biosynthesis of tumor necrosis factor alpha in human hepatoma cells involved the transcription factors ATF2 and c-Jun and stress-activated protein kinases. J Cell Biochem. 100(1), 242-255 (Epub ahead of print), (2006).
Beaupre D M and Kurzrock R. RAS and leukemia; from basic mechanisms to gene-directed therapy. J Clin Oncol 1999; 17: 1071-9.
Bederson J B, Pitts L H, Tsuji M, Nishimura M C, Davis R L, Bartkowski H. Rat middle cerebral artery occlusion: evaluation of the model and development of a neurologic examination. Stroke. 1986: 17: 472-476.
Berenblum I. A re-evaluation of the concept of co-carcinogenesis. Prog. Exp. Tumor Res. 11, 21-30 (1969).
Bezard et al., Effects of different schedules of MPTP administration on dopaminergic neurodegeneration in mice. Exp Neurol. 148: 288-92, 1997.
Boutwell R. K. Biochemical mechanism of tumor promotion, in mechanisms of tumor promotion and co-carcinogenesis. Eds. Slaga, T. J., Sivak. A. J. and Boutwell, R. K. Raven, N.Y., 49-58 (1978).
Boutwell R. K. The function and mechanism of promoters of carcinogensis. CRC Crit. Rev. Toxicol 2, 419-443 (1974).
Brooks A. I. et al. Paraquat elicited neurobehavioral syndrome caused by dopaminergic neuron loss Brain Res. 823, 1-10, 1999.
Brose N, Rosenmund C. Move over protein kinase C, you've got company: alternative effectors of diacylglycerol and phorbol esters. J Cell Sci; 115:4399-411 (2002).
Cancer Chemother Pharmacol. June; 57(6):789-95 (2006).
Cheson B D. Cassileth P A. Head D R, Schiffer C A, Bennett J M, Bloomfield C D, Brunning R, Gale R P, Grever M R, Keating M J, and et al. Report of the National Cancer Institute-sponsored workshop on definitions of diagnosis and response in acute myeloid leukemia. J Clin Oncol 1990; 8: 813-9.
Cui X X, Chang R L, Zheng X, Woodward D, Strair R, and Conney A H. A sensitive bioassay for measuring blood levels of 12-O-tetradecanoylphorbol-13-acetate (TPA) in patients: preliminary pharmacokinetic studies. Oncol Res 2002; 13: 169-74.
Deegan M. J., Maeda k. Differentiation of chronic lymphocytic leukemia cells after in vitro treatment with Epstein-Barr virus or phorbol ester. Immunologic and morphologic studies. Am J Hermatol. 17(4), 335-47 (1984).
Falcioni F., Rautmann A. et al. Influence of TPA (12-O-tetradodecanoyl-phorbol-13-acetate) on human B lymphocyte function. Clin Exp Immunol. 62(3), 163-2 (1985).
Farrell B, Godwin J, Richards S, Warlow C, et al. (1991). “The United Kingdom transient ischaemic attack (UK-TIA) aspirin trial: final results.” J Neurol Neurosurg Psychiatry 54 (12): 1044-1054.
Forbes I. J., Zalewski P. D., Letarte M. Human B-lymphocyte maturation sequence revealed by TPA-induced differentiation of leukaemia cells. Immunobiology 163(1), 1-6(1982).
Friedemann & Gerhardt. Neurobiol Aging, Regional effects of aging on dopaminergic function in the Fischer-344 rat. 13: 325-32, 1992.
Fujisawa K., Nasa K. et al. Production of interleukin (IL)-6 and IL-8 by a chorio-carcinama cell line, BeWo. Placenta 21(4). 354-60 (2000).
Gunjan Goel, Harinder P. S. Makkar. George Francis, and Klaus Becker. Phorbol Esters: Structure, Biological Activity, and Toxicity in Animals. International Journal of Toxicology, 2:279-288, 2007. Gogusev J., Barbey S., Nezelof C. Regulation of TNF-alpha and IL-I gene expression during TPA-induced differentiation of “Malignant histiocyosis” DEL cell line t(5:6) (q35:P21). Anticancer Res. 16(1), 455-60 (1996).
Granger C V, Devis L S, Peters M C, Sherwood C C, Barrett J E. Stroke rehabilitation: analysis of repeated Barthel Index measures. Arch Phys Med Rehabil. 1979; 60:14-17.
Hamburger, A. W., and Salmon, S. E. Primary bioassay of human tumor stem cells. Science (Wash. D.C.), 797: 461-463, 1977.
Han Z T, Zhu X X, Yang R Y, Sun J Z, Tian G F, Liu X J, Cao G S, Newmark H L, Conney A H, and Chang R L. Effect of intravenous infusions of 12-O-tetradecanoylphorbol-13-acetate (TPA) in patients with myelocytic leukemia: preliminary studies on therapeutic efficacy and toxicity. Proc Natl Acad Sci USA 1998; 95: 5357-61.
Han Z. T., Tong Y. K., He L. M., Zhang Y., Sun J. Z., Wang T. Y., Zhang H., Cui Y. L., Newmark H. L., Conney A. H., Chang R. L. 12-O-Tetradecanoyl-phorbol-13-acetate (TPA)-induced increase in depressed white blood cell counts in patients treated with cytotoxic cancer chemotherapeutic drugs. Proc. Natl. Acad. Sci. 95, 5363-5365 (1998).
Han Z. T., Zhu X. X., Yang R. Y., Sun J. Z., Tian G. F., Liu X. J., Cao G. S., Newmark H. L., Conney A. H., and Chang R. L. Effect of intravenous infusion of 12-O-tetradecanoyl-phorbol-13-acetate (TPA) in patients with myelocytic leukemia: preliminary studies on therapeutic efficacy and toxicity. Pro. Natl. Acad. Sci. 95, 5357-5361 (1998).
Harada S. et al.: Tumor Promoter. TPA. Enhances Replication of HTLV-III/LAV. Virology 154, 249-258 (1986).
Hecker E. In handbuch der allgemeinen patholgie, ed. Grundmann, E. (Springer-Verlag, Berlin-Heideiberg. Vol. IV 16, 651-676 (1975).
Hecker E. Structure-activity relationships in deterpene esters irritant and co-carcinogenic to mouse skin, in mechanisms of tumor promotion and co-carcinogenesis. Eds. Slaga. T. J., Sevak. A. j. and Boutwell. R. K. Raven. N.Y. 11-49 (1978).
Hofmann J. The potential for isoenzyme-selective modulation of protein kinase C. FASEB J. 11, 649-669 (1997).
Huberman E., Callaham M. F. Induction of terminal differentiation in human promyelocytic leukemia cells by tumor-promoting agents. Proc. Natl. Acad. Sci. 76, 1293-1297 (1979).
Hunter T. Signaling 2000 and beyond. Cell 100, 113-117 (2000).
Jordan C T. Unique molecular and cellular features of acute myelogenous leukemia stem cells. Leukemia 2002; 16: 559-62.
Kassel O, Sancono A, Kratzschmar J, Kreft B, Stassen M, and Cato A C. Glucocorticoids inhibit MAP kinase via increased expression and decreased degradation of MKP-1. Embo J 2001; 20: 7108-16.
Kazanietz M. G. Eyes Wide Shut: protein kinase C isoenzymes are not the only receptors for the phorbol ester tumor promoters. Mol. Carcinog. 28, 5-12 (2000).
Keoffler H. P., Bar-Eli M., Territo M. C. Phorbol ester effect on differentiation of human myeloid leukemia cells lines blocked at different stages of maturation. Cancer Res. 41, 919-926 (1981).
Kim S C, Hahn J S, Min Y H, Yoo N C, Ko Y W, and Lee W J. Constitutive activation of extracellular signal-regulated kinase in human acute leukemias: combined role of activation of MEK, hyperexpression of extracellular signal-regulated kinase, and downregulation of a phosphatase, PAC1. Blood 1999; 93: 3893-9.
Kiyoi H, Naoe T, Nakano Y, Yokota S, Minami S, Miyawaki S, Asou N, Kuriyama K, Jinnai I, Shimazaki C, Akiyama H, Saito K, Oh H, Motoji T, Omoto E, Saito H, Ohno R, and Ueda R. Prognostic implication of FLT3 and N-RAS gene mutations in acute myeloid leukemia. Blood 1999; 93: 3074-80.
Kobayashi M., Okada N. et al. Intracellular interleukin-1 alpha production in human gingival fibroblasts is differentially regulated by various cytokines. J Dent Res. 78(4), 840-9 (1999).
Koeffler H. P. Phorbol diester-induced macrophage differentiation of leukemic blasts from patients with human myelogenous leukemia. J. Clin. Invest. 66, 1101-1108 (1980).
Kudo M., Aoyama A., Ichimori S. and Fukunaga N. An animal model of cerebral infarction: homologous blood clot emboli in rats. Stroke 13: 505-508 (1982)
Lebien T. W., Bollum F. J. et al. Phorbol ester-induced differentiation of a non-T, non-B leudemic cell line: model for human lymphoid progenitor cell development. J Immunol. 128(3), 1316-20 (1982).
McCornack A. L. et al. Environmental risk factors and Parkinson's disease: selective degeneration of nigral dopaminergic neurons caused by the herbicide paraquat. Neurobiol Dis. 10, 119-127, 2002.
Meinhardt G., Roth J., Hass R. Activation of protein kinase C relays distinct signaling pathways in the same cell type: differentiation and caspase-mediated apoptosis. Cell Death Differ. 7, 795-803 (2000).
Milella M, Kornblau S M, Estrov Z, Carter B Z, Lapillonne H, Harris D, Konopleva M, Zhao S, Estey E, and Andreeff M. Therapeutic targeting of the MEK/MAPK signal transduction module in acute myeloid leukemia. J Clin Invest 2001; 108: 851-9.
Mochty-Rosen D., Kauvar L. M. Modulating protein kinase C signal transduction. Adv. Pharmacol. 44, 91-145 (1998).
Morgan M A, Dolp O, and Reuter C W. Cell-cycle-dependent activation of mitogen-activated protein kinase kinase (MEK-1/2) in myeloid leukemia cell lines and induction of growth inhibition and apoptosis by inhibitors of RAS signaling. Blood 2001; 97: 1823-34.
Nagasawa K., Chechgik B. E. et al. Modulation of human T-cell differentiation markers by 12-O-tetradecanoylphorbal-13-acetate. Thymus. 3(4-5), 307-18, (1981).
Nakao Y., Matsuda S. et al. Paradoxical anti-leukemic effects of plant-derived tumor promoters on a human thymic lymphoblast cell line. Int J Cancer 30(6), 687-95 (1982).
Nakao Y., Matsuda S. et al. Phorbol ester-induced differentiation of human T-lymphoblastic cell line HPB-ALL. Cancer Res. 42(9), 33843-50 (1982).
Newton A. C. Protein kinase C: structure, function and regulation. J. Biol. Chem. 270, 28495-28499 (1995).
Niederman T. M. J., Ratner L. et al. Human Immunodeficiency Virus Type I Nef Protein Inhibits NF-KB Induction in Human T Cells. J. Virology 66 (10), 6313-6219 (1992).
Norwell P., Shankey T. V. et al. Proliferation, differentiation and cytogenetics of chronic leukemic B lymphocytes cultured with mitomycin-treated normal cells. Blood 57(3), 444-51 (1981).
O'Banion M. K., Miller J. C. et al. Interleukin-1 beta induces prostaglandin G/H synthase-2 (cyclooxygenase-2) in primary murine astrocyte cultures. J Neurochem 66(6), 2532-40 (1996).
Okamura J., Geffind E. W., Letarte M. Heterogeneity of the response of chronic lymphocytic leukemia cells to phorbol ester. Blood 60(5). 1082-8 (1982).
Palombella V J, Rando O J, Goldberg A L, and Maniatis T. The ubiquitin-proteasome pathway is required for processing the NF-kappa B I precursor protein and the activation of NF-kappa B. Cell 1994; 78: 773-85.
Platanias L C. Map kinase signaling pathways and hematologic malignancies. Blood 2003; 101:4667-79.
Polliack A., Leizerowitz R., Korkesh A., Gurfel D., Gamliel H., Galili L. Exposure to TPA in vitro as an aid in the classification of blasts in human myelogenous and lymphoid leukemias. Am. J. Hematol. 13, 199-211 (1982).
Redondo P., Garci-Foncillas J. et al. Differential modulation of IL-8 and TNF-alpha expression in human keratinocytes by buffomedil chlorhydrate and pentoxifylline. Exp. Dermatol. 6(4), 186-94 (1997).
Rovera G., Santoli D., Damsky C. Human promyelocytic cells in culture differentiate into macrophage-like cells treated with a phorbol diester. Pro. Natl. Acad. Sci. 7, 2779-2783 (1979).
Rullas J., Alcami J. et. al. Receptors in peripheral blood lymphocytes. Antivir. Ther. 9 (4). 545-554 (2004).
Rwigema J C, Beck B, Wang W, Doemling A, Epperly M W, Shields D, Goff J P, Franicola D, Dixon T, Frantz M C, Wipf P, Tyurina Y, Kagan V E, Schaar D, Goodell L, Aisner J, Cui X X, Han Z T, Chang R, Martin J, Grospe S, Dudek L, Riley J, Manago J, Lin Y, Rubin E H, Conney A, Strair R K. A phase I clinical trial of 12-O-tetradecanoylphorbol-13-acetate for patients with relapsed/refractory malignancies.
Scheinman R I, Cogswell P C, Lofquist A K, and Baldwin A S. Jr. Role of Transcriptional Activation of IkappaBalpha in Mediation of Immunosuppression by Glucocorticoids. Science 1995; 270: 283-286.
Shkolnick T., Schlossman S. F., Griffin J. D. Acute undifferentiated leukemia: induction of partial differentiation by phorbol ester. Leuk. Res. 9, 1-17 (1985).
Shwarz M. et. al. High-level IL-10 production by monoclonal antibody-stimulated human T cells. Immunology 86, 364-371 (1995).
Staber P B, Linkesch W, Zauner D, Beham-Schmid C, Guelly C, Schauer S, Sill H, and Hoefler G. Common alterations in gene expression and increased proliferation in recurrent acute myeloid leukemia. Oncogene 2004; 23: 894-904.
Steube K. G., Meyer C., Drexler H. G. Constitutive excretion of hematopoietic cytokines by human carcinoma cell lines and its up-regulation by interleukin-1 and phorbol ester. Oncol. Rep. 6(20), 427-32 (1999).
Strair R K, Schaar D, Goodell L, Aisner J, Chin K V, Eid J, Senzon R, Cui X X, Han Z T, Knox B, Rabson A B, Chang R, and Conney A. Administration of a phorbol ester to patients with hematological malignancies: preliminary results from a phase I clinical trial of 12-O-tetradecanoylphorbol-13-acetate. Clin Cancer Res 2002; 8: 2512-8
Tanner, C. M., Kamel, F., Ross, G. W. Hoppin, J. A., Goldman, S. M., Korell, M., Marras, C., Bhudhikanok, G. S., Kasten, M., Chade, A. R. Comyns, K., Richards, M. B., Meng, C., Priestley, B., Fernandez, H. H., Cambi, F., Umbach, D. M., Blair, A., Sandier, D. P., and Langston, J. W., Rotenone, Paraquat and Parkinson's Disease, 2011, Environmental Health Perspectives, 119; 866-872.
Thiruchelvam M. et al. Developmental exposure to the pesticides paraquat and maneb and the Parkinson's disease phenotype. Neurotoxicology 23, 621-633, 2002.
Totterman T. H., Nilsson K., Sundstrom C. Phorbol ester-induced differentiation of chronic lymphoctic leukaemia cells. Nature 288(5787), 176-8 (1980)
Towatari M, Iida H, Tanimoto M, Iwata H, Hamaguchi M, and Saito H. Constitutive activation of mitogen-activated protein kinase pathway in acute leukemia cells. Leukemia 1997; 11: 479-84.

Ungerstedt, U. and G. W. Arbuthnott, Quantitative recording of rotational behavior in rats after 6-hydroxy-dopamine lesions of the nigrostriatal dopamine system. Brain Res, 1970. 24(3): p. 485-93.
Van Duuren, B. L. Tumor-promoting agents in two-stage carcinogenesis. Prog. Exp. Tumor Res. 11, 31-68 (1969).
Varastet et al., Chronic MPTP treatment reproduces in baboons the differential vulnerability of mesencephalic dopaminergic neurons observed in Parkinson's disease. Neuroscience, 63: 47-56.1994.
Yamamoto Y, Kiyoi H, Nakano Y, Suzuki R. Kodera Y, Miyawaki S, Asou N, Kuriyama K, Yagasaki F, Shimazaki C, Akiyama H, Saito K, Nishimura M, Motoji T, Shinagawa K, Takeshita A, Saito H, Ueda R, Ohno R, and Naoe T. Activating mutation of D835 within the activation loop of FLT3 in human hematologic malignancies. Blood 2001; 97: 2434-9.
YIP, Y. K. et al. Stimulation of human gamma interferon production by diterpene esters. Infection and Immunity 34(1) 131-139 (1981).
Zhao J., Sharma Y., Agarwal R. Significant inhibition by the flavonoid antioxidant silymarin against 12-O-tetradecanoylphorbol 13-acetate-caused modulation of antioxidant and inflammatory enzymes and cyclooxygenase2 and interleukin-1 alpha expression in SENCAR mouse epidermis: implications in the prevention of stage I tumor promotion. Mol Carcinog. 26(4), 321-33 (1999).

We claim:

1. A method for treating or preventing one or more of the symptoms of Parkinson's disease in a mammalian subject comprising administering an effective amount of phorbol ester or derivative compound of Formula I, or a pharmaceutically-acceptable salt, isomer, enantiomer, solvate, hydrate, polymorph or prodrug thereof to said subject 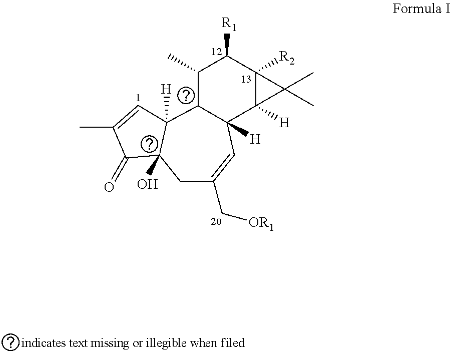
Wherein R₁ and R₂ are selected from the group consisting of hydrogen, hydroxyl, 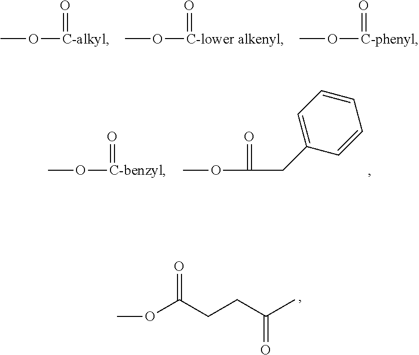
and substituted derivatives thereof; R₃ is selected from hydrogen, 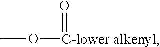
and substituted derivatives thereof.

2. The method of claim 1, wherein R₁ or R₂ is 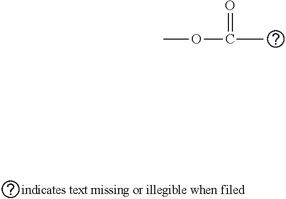
the remaining R₁ or R₂ is 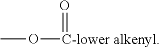
and R₃ is hydrogen.

3. The method of claim 1, wherein the phorbol ester is phorbol 13-butyrate, phorbol 12-decanoate, phorbol 13-decanoate, phorbol 12,13-diacetate, phorbol 13,20-diacetate, phorbol 12,13-dibenzoate, phorbol 12,13-dibutyrate, phorbol 12,13-didecanoate, phorbol 12,13-dihexanoate, phorbol 12,13-dipropionate, phorbol 12-myristate, phorbol 13-myristate, phorbol 12,13,20-triacetate, 12-deoxyphorbol 13-angelate, 12-deoxyphorbol 13-angelate 20-acetate, 12-deoxyphorbol 13-isobutyrate, 12-deoxyphorbol 13-isobutyrate-20-acetate, 12-deoxyphorbol 13-phenylacetate, 12-deoxyphorbol 13-phenylacetate 20-acetate, 12-deoxyphorbol 13-tetradecanoate, phorbol 12-tigliate 13-decanoate, 12-deoxyphorbol 13-acetate, phorbol 12-acetate, or phorbol 13-acetate.

4. The method of claim 1, wherein the phorbol ester is 12-O-tetradecanoylphorbol-13-acetate.

5. The method of claim 1, further comprising administering at least one secondary or adjunctive therapeutic agent that is effective in a combinatorial formulation or coordinate treatment regimen with said phorbol ester or derivative compound of Formula I to treat or prevent one or more of the symptoms of Parkinson's disease.

6. The method of claim 1, wherein the at least one secondary or adjunctive therapeutic agent is administered to said subject in a coordinate administration protocol, simultaneously with, prior to, or after, administration of said phorbol ester to said subject.

7. The method of claim 4, wherein the at least one secondary or adjunctive therapeutic agent is carbidopa/levodopa, pyridoxine, selegiline, rasagiline, tolcapone, dopamine agonists, MAO-B inhibitors, amantadine, or anticholinergics.

8. The method of claim 1, further comprising surgical intervention in combination with phorbol ester or derivative compound of Formula I to treat or prevent symptoms of Parkinson's disease in said subject.

9. The method of claim 7, wherein the surgical intervention is deep brain stimulation or lesion formation.

10. The method of claim 1, wherein the one or more symptoms of Parkinson's disease is tremor at rest, stiffness, bradykinesia, rigidity, speech impairment, cognitive impairment, dementia, mood impairment, drowsiness, insomnia and postural instability.

11. The method of claim 1, wherein said effective amount comprises between about 10 μg and about 1500 μg of said phorbol ester or derivative compound of Formula I every day.

12. The method of claim 1, wherein said effective amount comprises between about 125 μg to about 500 μg of said phorbol ester or derivative compound of Formula I every day.

13. A composition for treating Parkinson's disease in a mammalian subject comprising an effective amount of a phorbol ester of Formula I, or a pharmaceutically-acceptable salt, isomer, or enantiomer thereof 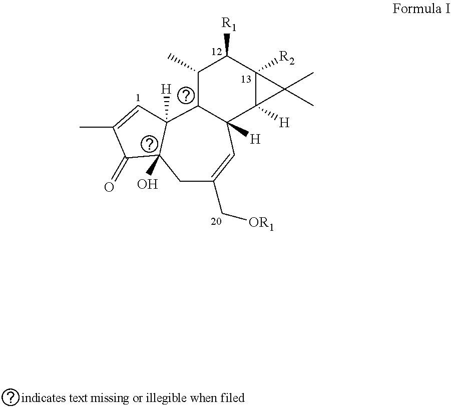
Wherein R₁ and R₂ are selected from the group consisting of hydrogen, hydroxyl, 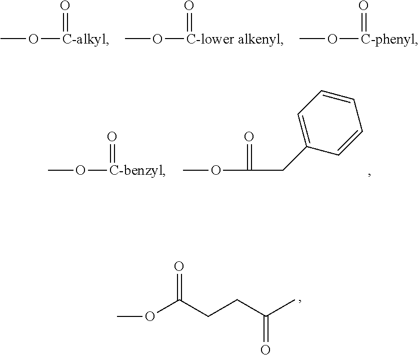
and substituted derivatives thereof, R₃ is hydrogen, 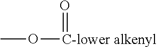
and substituted derivatives thereof;
and at least one secondary or adjunctive therapeutic agent that is effective in a combinatorial formulation with said phorbol ester or derivative compound of Formula I to treat Parkinson's disease in said subject.

14. The composition of claim 14, wherein R₁ or R₂ is 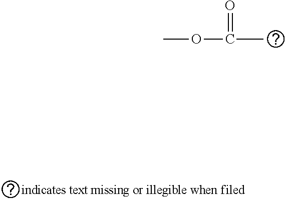
the remaining R₁ or R₂ is 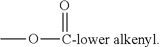
and R₃ is hydrogen.

15. The composition of claim 14, wherein the phorbol ester is phorbol 13-butyrate, phorbol 12-decanoate, phorbol 13-decanoate, phorbol 12,13-diacetate, phorbol 13,20-diacetate, phorbol 12,13-dibenzoate, phorbol 12,13-dibutyrate, phorbol 12,13-didecanoate, phorbol 12,13-dihexanoate, phorbol 12,13-dipropionate, phorbol 12-myristate, phorbol 13-myristate, phorbol 12,13,20-triacetate, 12-deoxyphorbol 13-angelate, 12-deoxyphorbol 13-angelate 20-acetate, 12-deoxyphorbol 13-isobutyrate, 12-deoxyphorbol 13-isobutyrate-20-acetate, 12-deoxyphorbol 13-phenylacetate, 12-deoxyphorbol 13-phenylacetate 20-acetate, 12-deoxyphorbol 13-tetradecanoate, phorbol 12-tigliate 13-decanoate, 12-deoxyphorbol 13-acetate, phorbol 12-acetate, or phorbol 13-acetate.

16. The composition of claim 14, wherein the phorbol ester is 12-O-tetradecanoylphorbol-13-acetate.

17. The composition of claim 14, wherein the at least one secondary or adjunctive therapeutic agent is selected from the group consisting of levodopa, carbidopa, pyridoxine, seleyiline, rasagiline, tolcapone, dopamine agonists, MAO-B inhibitors, amantadine, or anticholinergics.

18. The composition of claim 14, wherein the composition contains at least two secondary or adjunctive therapeutic agents.
